SEQUENCE LISTING (14 sequences)

SEQ ID NO: 1 - DNA, 58 bp, Artificial Sequence
  Partial sequence of Lefitolimod
cctaggggttaccaccttcattggaaaacgttcttcggggcgttcttaggtggtaacc

SEQ ID NO: 2 - DNA, 58 bp, Artificial Sequence
  Partial sequence of dSLIM 2006
aggtggtaacccctaggggttaccaccttcatcgtcgttttgtcgttttgtcgttctt

SEQ ID NO: 3 - DNA, 116 bp, Artificial Sequence
  Complete sequence of Lefitolimod
aaaacgttcttcggggcgttcttaggtggtaacccctaggggttaccaccttcattggaaaacgttcttcggggcgttcttaggtggtaacccctaggggttaccaccttcattgg

SEQ ID NO: 4 - DNA, 116 bp, Artificial Sequence
  Complete sequence of dSLIM 2006
tcatcgtcgttttgtcgttttgtcgttcttaggtggtaacccctaggggttaccaccttcatcgtcgttttgtcgttttgtcgttcttaggtggtaacccctaggggttaccacct

SEQ ID NO: 5 - DNA, 33 bp, Artificial Sequence
  TLR-9 agonist comprising at least one nucleotide in L-configuration
  misc_feature: Nucleotides 31 and 32 in L-configuration
ggggtcattaaaacgtgacgtgacgttcttttt

SEQ ID NO: 6 - DNA, 37 bp, Artificial Sequence
  TLR-9 agonist comprising at least one nucleotide in L-configuration
  misc_feature: Nucleotides 1, 2, 35 and 36 in L-configuration
ggggggggtcattaaaacgtgacgtgacgttcttttt

SEQ ID NO: 7 - DNA, 32 bp, Artificial Sequence
  TLR-9 agonist comprising at least one nucleotide in L-configuration
  misc_feature: Nucleotides 30 and 31 in L-configuration
ggggtcattaaacgtgacgtgacgttcttttt

SEQ ID NO: 8 - DNA, 34 bp, Artificial Sequence
  TLR-9 agonist comprising at least one nucleotide in L-configuration
  misc_feature: Nucleotides 32 and 33 in L-configuration
ggggtcattaaacgttcttcggggcgttcttttt

SEQ ID NO: 9 - DNA, 39 bp, Artificial Sequence
  TLR-9 agonist comprising at least one nucleotide in L-configuration
  misc_feature: Nucleotides 37 and 38 in L-configuration
ggggtcattaaatcgtcgttttgtcgttttgtcgttttt

SEQ ID NO: 10 - DNA, 21 bp, Artificial Sequence
  TLR-9 agonist comprising at least one nucleotide in L-configuration
  misc_feature: Nucleotides 1, 2, 19 and 20 in L-configuration
gggggacgatcgtcggggggt

SEQ ID NO: 11 - DNA, 33 bp, Artificial Sequence
  TLR-9 agonist comprising at least one nucleotide in L-configuration
  misc_feature: Nucleotides 31 and 32 in L-configuration
gggtcattaaacgttcttcggggcgttcttttt

SEQ ID NO: 12 - DNA, 38 bp, Artificial Sequence
  TLR-9 agonist comprising at least one nucleotide in L-configuration
  misc_feature: Nucleotides 1, 2, 36 and 37 in L-configuration
ggggggggtcattaaacgttcttcggggcgttcttttt

SEQ ID NO: 13 - DNA, 36 bp, Artificial Sequence
  TLR-9 agonist comprising at least one nucleotide in L-configuration
  misc_feature: Nucleotides 1, 2, 34 and 35 in L-configuration
ggggggggtcattaaacgtgacgtgacgttcttttt

SEQ ID NO: 14 - DNA, 43 bp, Artificial Sequence
  TLR-9 agonist comprising at least one nucleotide in L-configuration
  misc_feature: Nucleotides 1, 2, 41 and 42 in L-configuration
ggggggggtcattaaatcgtcgttttgtcgttttgtcgttttt

(19) United States
(12) Patent Application Publication
(10) Pub. No.: US 2021/0155933 A1
(43) Pub. Date: May 27, 2021

(54) TLR-9 AGONISTS FOR MODULATION OF TUMOR MICROENVIRONMENT

(71) Applicant: Gilead Sciences, Inc., Foster City, CA (US)

(72) Inventors: Manuel SCHMIDT, Berlin (DE); Kerstin KAPP, Berlin (DE)

(21) Appl. No.: 16/642,649

(22) PCT Filed: Aug. 31, 2018
(86) PCT No.: PCT/EP2018/073534

§ 371 Date: Feb. 27, 2020

(30) Foreign Application Priority Data
Aug. 31, 2017 (EP) 17001465.8
Jan. 15, 2018 (EP) 18151734.3

(51) Int. Cl. C12N 15/117; A61K 9/00; A61K 45/06; A61K 9/08; A61K 47/26; A61P 35/00

(52) U.S. Cl. CPC C12N 15/117; A61K 9/0019; A61K 45/06; A61K 9/08; C12N 2310/531; A61P 35/00; C12N 2310/17; C12N 2320/31; A61K 47/26

(57) ABSTRACT

A TLR-9 agonist for use in the treatment of a tumor disease, in particular of colon cancer, and for the modulation of the tumor microenvironment.

### FIELD OF THE INVENTION

The present invention relates to the field of tumor therapy, in particular to the field of treatment of solid tumors, more particular to the treatment of colon cancer in humans. The invention further relates to the field of TLR-9 agonists and their use in the treatment of said tumor diseases. In yet a further aspect of the invention it relates to the field of modulating the tumor microenvironment of tumors, in particular of solid tumors, and thereby supporting tumor therapy.

### BACKGROUND OF THE INVENTION

The tumor microenvironment (TME) is the cellular environment in which the tumor exists, including surrounding blood vessels, immune cells, fibroblasts, bone marrow-derived inflammatory cells, lymphocytes, signaling molecules and the extracellular matrix. The tumor and the surrounding microenvironment are closely related and interact constantly. Tumors can influence the microenvironment by releasing extracellular signals, promoting tumor angiogenesis and inducing peripheral immune tolerance, while the immune cells in the microenvironment can affect the growth and evolution of cancerous cells.

Historically, the discovery of melanoma-specific T cells in patients led to the strategy of adoptively transferring large numbers of in vitro-expanded tumor-infiltrating lymphocytes (TILs) which has proven that the immune system has the potential to control cancer. However, adoptive T cell therapy (ACT) with TILs has not had the success of ACT with virus-specific CD8+ T cells. The TME of solid cancers appears to be fundamentally different to that of the leukemias, in which clinical ACT trials with chimeric antigen receptor T cells have demonstrated efficacy.

Tumor infiltrating lymphocytes (TILs) are lymphocytes that penetrate a tumor (i.e infiltrate into the tumor). Preclinical mice studies implicated that T cell accumulation near cancer cells is restricted. Overcoming this restriction revealed enhanced antitumor effects. T cells reach tumor sites via the circulatory system. The TME appears to preferentially recruit other immune cells over T cells from that system. One such mechanism is the release of cell-type specific chemokines. Another is the TME's capacity to posttranslationally alter chemokines. Hence, it is known by the skilled person that TILs are implicated in killing tumor cells and that the presence of lymphocytes in tumors is often associated with better clinical outcomes.

Tumor-associated macrophages (TAMs) originate mainly from the peritumoral tissue or bone marrow and can be divided into two main types: M1 and M2. In particular, M1 macrophages have anti-tumoral properties. Therefore, either a conversion of M2 macrophages into M1 macrophages or a preferential infiltration of M1 macrophages is beneficial for an anti-tumor immune response.

Hence, there are further needs to identify means and methods to modulate the TME in order to improve tumor therapy. In particular, there is a need for further improved methods and measures to stimulate the infiltration of TILs, in particular of T cells, such as CD3+ T cells, and of macrophages into a tumor and thereby support tumor therapy.

### DESCRIPTION OF THE INVENTION

In one embodiment, this objective is solved by providing a TLR-9 agonist for use in the treatment of a tumor disease, preferably of colon cancer, in a subject in need thereof, said TLR-9 agonist comprises
i. an oligodeoxyribonucleotide comprising at least one unmethylated CG dinucleotide, wherein C is deoxycytidine and G is deoxyguanosine and
ii. at least one stretch of at least three, in particular of four, consecutive deoxyguanosines,
and the deoxyribose moieties of the oligodeoxyribonucleotide are linked by phosphodiester bonds, wherein the treatment with said TLR-9 agonist stimulates the infiltration of CD3+ T cells into the tumor.

In a preferred embodiment, the infiltrating CD3+ T cells are CD4+ or CD8+ T cells, in a more preferred embodiment CD8+ T cells. In another preferred embodiment, the treatment with said TLR-9 agonist leads to an increased frequency of cytotoxic effector T cells (CD8+CD69+ Granzyme (Grz) B+) within the CD8+ T cell population in the tumor. The population of cytotoxic effector T cells (CD8+CD69+ Granzyme B+) may also be termed activated CD8+ T cells with cytolytic potential, i.e. this cell population preferentially has anti-tumorigenic potential.

In another preferred embodiment, the treatment with said TLR-9 agonist leads to an increased ratio of CD8+ T cells, preferably of cytotoxic effector T cells (CD8+CD69+ Granzyme B+), to regulatory T cells. Regulatory T cells have pro-tumorigenic potential.

Regulatory T cells can suppress the anti-tumor effects of T cells e.g. by secretion of inhibitory cytokines, deprivation of IL-2 or by inhibitory molecules expressed on their surface (e.g. CTLA-4).

Therewith, one objective is that the treatment with said TLR-9 agonist is for stimulating the infiltration of CD3+ T cells into the tumor. In a preferred embodiment, the treatment with said TLR-9 agonist is for increasing the frequency of cytotoxic effector T cells (CD8+CD69+ Granzyme (Grz) B+) within the CD8+ T cell population in the tumor and/or for increasing the ratio of CD8+ T cells, preferably of cytotoxic effector T cells (CD8+CD69+ Granzyme B+), to regulatory T cells.

In one embodiment, the tumor is infiltrated in its periphery and/or its center, preferably in its center.

Therewith, one objective is that the treatment with said TLR-9 agonist is for infiltrating the tumor in its periphery and/or its center, preferably in its center.

In one embodiment, the tumor is a solid tumor, preferably colon cancer, and the subject to be treated is a human.

The objective of the present invention is further solved by applying a method for increasing the infiltration of CD3+ T cells, preferably CD8+ T cells, into a tumor, preferably colon cancer, comprising administering a TLR-9 agonist to a patient in need thereof, wherein the TLR-9 agonist comprises
i. an oligodeoxyribonucleotide comprising at least one unmethylated CG dinucleotide, wherein C is deoxycytidine and G is deoxyguanosine and
ii. at least one stretch of at least three, in particular of four, consecutive deoxyguanosines and
wherein the deoxyribose moieties of the oligodeoxyribonucleotide are linked by phosphodiester bonds.

In another embodiment, said method serves for increasing the frequency of cytotoxic effector T cells (CD8+CD69+ Granzyme B+) within the CD8+ T cell population in the tumor.

In another embodiment, said method serves for increasing the ratio of CD8+ T cells, preferably of cytotoxic effector T cells (CD8+CD69+ Granzyme B+), to regulatory T cells.

The inventors have found that such TLR-9 agonists can lead to an increased trafficking of T cells to the tumor. Therewith the local concentration of T-cells in the TME and/or the tumor raises, i.e. the T-cells accumulate in the close proximity to the tumor and infiltrate there into. The restriction of T-cells by the TME thus seems to be overcome. Accordingly the T-cells can exhibit their anti-tumor effects. Thus, in one specific aspect of the invention the TLR-9 agonists are used to increase the efficacy of CD3+ T cells, preferably CD8+ T cells.

In another embodiment, the objective of the present invention is solved by providing a TLR-9 agonist for use in the treatment of a tumor disease, preferably of colon cancer, in a subject in need thereof, said TLR-9 agonist comprises
i. an oligodeoxyribonucleotide comprising at least one unmethylated CG dinucleotide, wherein C is deoxycytidine and G is deoxyguanosine and
ii. at least one stretch of at least three, in particular of four, consecutive deoxyguanosines,

and the deoxyribose moieties of the oligodeoxyribonucleotide are linked by phosphodiester bonds, wherein the treatment with said TLR-9 agonist stimulates the infiltration of macrophages, preferably M1 macrophages, into the tumor and/or the treatment with said TLR-9 agonist leads to an increased ratio of M1 macrophages to M2 macrophages within the tumor.

Therewith, one objective is that the treatment with said TLR-9 agonist is for stimulating the infiltration of macrophages, preferably M1 macrophages, into the tumor and/or the treatment with said TLR-9 agonist is for increasing the ratio of M1 macrophages to M2 macrophages within the tumor.

Preferably, the infiltration of the tumor by the CD3+ T cells and/or by the macrophages, preferably M1 macrophages, is stimulated compared with the infiltration without the treatment of the TLR-9 agonist and/or the ratio of M1 macrophages to M2 macrophages within the tumor is increased compared to the ratio of M1 macrophages to M2 macrophages without the treatment of the TLR-9 agonist.

In another embodiment, the objective of the present invention is solved by applying a method for increasing the infiltration of macrophages, preferably M1 macrophages, into a tumor, preferably colon cancer, and/or for increasing a ratio of M1 macrophages to M2 macrophages within a tumor, preferably colon cancer, comprising administering a TLR-9 agonist to a patient in need thereof, wherein the TLR-9 agonist comprises
i. an oligodeoxyribonucleotide comprising at least one unmethylated CG dinucleotide, wherein C is deoxycytidine and G is deoxyguanosine and
ii. at least one stretch of at least three, in particular of four, consecutive deoxyguanosines and
wherein the deoxyribose moieties of the oligodeoxyribonucleotide are linked by phosphodiester bonds.

In yet another particularly preferred embodiment of the invention the TLR-9 agonists as defined in above are used to convert so called “cold tumors”—i.e. tumors with a low degree of immune cell infiltration—into “hot tumors”, which are immunogenic tumors, i.e. with an intermediate or high degree of immune cell infiltration. The concept of “cold” and “hot” tumors is well known to the person skilled in the art. Cold tumors are usually enriched in immunosuppressive cytokines and have high numbers of Treg cells and myeloid-derived suppressor cells (MDSC). Cold tumors usually have few numbers of T_{H}1 cells, NK cells and CD8+ T cells and few functional antigen-presenting cells (APC) (for example dendritic cells/DC). In contrast, hot tumors are enriched in T_{H}1-type chemokines and have high numbers of effector immune cells (T_{H}1 cells, NK cells and CD8+ T cells) and high numbers of DC.

The degree of immune cell infiltration can for example be measured by the so called “Immunoscore”, which is used to predict clinical outcome in patients with cancer. The consensus Immunoscore is a scoring system to summarise the density of CD3+ and CD8+ T-cells within the tumor and its invasive margin. For example, the Immunoscore can be classified as low, intermediate and high depending on the CD3+/CD8+ T cell density, whereas a 0-25% density is preferably scored as low, a 25-70% density is preferably scored as intermediate and a 70-100% density is preferably scored as high (cf. Pages F. et al. (2018) Lancet 391(10135):2128-2139) in a reference study. Cold tumors are defined as having a low degree of immune cell infiltration, i. e. preferably have a low Immunoscore. Hot tumors are defined as having an intermediate or high degree of immune cell infiltration, i. e. preferably have an intermediate or high Immunoscore.

Some tumor types belong to the type of hot tumors even before treatment, for example melanoma. These tumors usually respond well to drugs, such as checkpoint inhibitors. The present invention suggests that administration of a TLR-9 agonist in these tumors leads to a further enhanced activation of T cells and infiltration of immune cells into the tumor. The treatment with a TLR-9 agonist may also lead to an enhanced responsiveness of the tumor to other drugs, for example checkpoint inhibitors.

Cold tumors usually do not respond well to drugs such as checkpoint inhibitors. The present invention suggests that a TLR-9 agonist can improve such responsiveness by increasing the infiltration of immune cells into the tumor and thereby positively influencing the TME. Patients with cold tumors therefore especially benefit from the treatment with a TLR-9 agonist. The tumor may as a result show better responsiveness to drugs, such as checkpoint inhibitors. Cold tumors may thereby be converted into hot tumors.

An especially preferred TLR-9 agonist for this particular use is Lefitolimod.

In one embodiment, a use of a TLR-9 agonist is provided for increasing the infiltration of CD3+ T cells, preferably CD8+ T cells, into a tumor, preferably colon cancer, and/or for increasing the frequency of cytotoxic effector T cells (CD8+CD69+ Granzyme B+) within the CD8+ T cell population in the tumor and/or for increasing the ratio of CD8+ T cells, preferably of cytotoxic effector T cells (CD8+CD69+ Granzyme B+), to regulatory T cells and/or for increasing the infiltration of macrophages, preferably M1 macrophages, into the tumor and/or for increasing a ratio of M1 macrophages to M2 macrophages within the tumor, wherein the TLR-9 agonist comprises
i. an oligodeoxyribonucleotide comprising at least one unmethylated CG dinucleotide, wherein C is deoxycytidine and G is deoxyguanosine and
ii. at least one stretch of at least three, in particular of four, consecutive deoxyguanosines and

wherein the deoxyribose moieties of the oligodeoxyribonucleotide are linked by phosphodiester bonds.

The administration of a TLR-9 agonist as of the invention thus leads to a reduction of the tumor growth rate. This could be demonstrated by the inventors in a murine colon carcinoma model (CT26) as described in more detail in the example section below.

In a preferred embodiment of the invention the TLR-9 agonist can be administered directly into the tumor, i.e. intratumorally, or subcutaneously. A subcutaneous injection is preferably administered into the subcutis, the layer of skin directly below the dermis and epidermis, collectively referred to as the cutis. To mimic a subcutaneous application in humans, as demonstrated in the example section below, mice were treated subcutaneously at a distant site far from the draining lymph area of the inoculated tumor. An intratumoral injection is administered directly into the tumor, preferably into the center of the tumor.

Subcutaneous injection is possible for many tumor types and can also be used to target metastasizing tumors. For the intratumoral injection, the tumor is preferably non-metastasized and easily accessible, for example melanoma or head and neck squamous-cell carcinoma (HNSCC). Liver metastases, however, can also be targeted by intratumoral injection, preferably by image-assisted injection. Subcutaneous injection can be repeatedly used over a longer time period. Intratumoral injection allows a lower dosis compared with subcutaneous injection because the TLR-9 agonist is directly applied into the tumor. The TLR-9 agonists of the present invention, preferably Lefitolimod, can be applied at high doses due to their low toxicity as a result of the lack of phosphorothioates.

Toll-like receptors (TLRs) are known to the person skilled in the art. They are present on many cells of the immune system and have been shown to be involved in the innate immune response. In vertebrates, this family consists of eleven proteins called TLR-1 to TLR-11 that are known to recognize pathogen associated molecular patterns from bacteria, fungi, parasites, and viruses.

TLRs are a key means by which vertebrates recognize and mount an immune response to foreign molecules and also provide a means by which the innate and adaptive immune responses are linked ((Akira, S. et al. (2001) Nature Immunol. 2:675-680; Medzhitov, R. (2001) Nature Rev. Immunol. 1: 135-145)). Some TLRs are located on the cell surface to detect and initiate a response to extracellular pathogens and other TLRs are located inside the cell to detect and initiate a response to intracellular pathogens.

TLR-9 is known to recognize unmethylated CpG motifs in bacterial DNA and in synthetic oligonucleotides (Hemmi, H. et al. (2000) Nature 408:740-745). Naturally occurring agonists of TLR-9 have been shown to produce anti-tumor activity (e.g. tumor growth and angiogenesis) resulting in an effective anti-cancer response (e.g. anti-leukemia) (Smith, J. B. and Wickstrom, E. (1998) J. Natl. Cancer Inst. 90: 1 146-1 154).

The TLR-9 agonists of the present invention comprise an oligodeoxyribonucleotide comprising at least one unmethylated CG dinucleotide, wherein C is deoxycytidine and G is deoxyguanosine and at least one stretch of at least three, in particular of four, consecutive deoxyguanosines, wherein the deoxyribose moieties of the oligodeoxyribonucleotide are linked by phosphodiester bonds.

Preferred TLR-9 agonists comprise an oligodeoxyribonucleotide comprising at least one unmethylated CG dinucleotide, wherein C is deoxycytidine and G is deoxyguanosine and at least one stretch of at least three, in particular of four, consecutive deoxyguanosines, and the deoxyribose moieties of the oligodeoxyribonucleotide are linked by phosphodiester bonds, wherein the at least one CG dinucleotide is part of a sequence N¹N²CGN³N⁴, wherein N¹N² is AA, TT, GG, GT, GA or AT and N³N⁴ is CT, TT, TG or GG and C is deoxycytidine, G is deoxyguanosine, A is deoxyadenosine, and T is deoxythymidine.

In one embodiment, the oligodeoxyribonucleotide comprises at least one nucleotide in L-configuration, preferably the at least one nucleotide in L-configuration is comprised within the terminal five nucleotides of at least one end of the oligodeoxyribonucleotide, preferably within the terminal five nucleotides of the 3′ end of the oligodeoxyribonucleotide.

In one embodiment, the TLR-9 agonist comprising at least one nucleotide in L-configuration has one or more of the following features:
the at least one stretch of at least three, in particular of four, consecutive deoxyguanosines is located at the 5′ end of the oligodeoxyribonucleotide,
at least one stretch of at least three consecutive deoxyguanosines is located between two CG dinucleotides,
one stretch of at least three, in particular of five, consecutive deoxythymidines is located at the 3′ end of the oligodeoxyribonucleotide,
at least one of the three 3′ terminal and/or 5′ terminal deoxynucleotides is in L-configuration,
two of the three 3′ terminal deoxynucleotides are in L-configuration,
the first and the second 5′ terminal deoxynucleotide are in L-configuration.

In a particularly preferred embodiment, in the TLR-9 agonist comprising at least one nucleotide in L-configuration
the at least one stretch of at least three, in particular of four, consecutive deoxyguanosines is located at the 5′ end of the oligodeoxyribonucleotide, and
two of the three 3′ terminal deoxynucleotides are in L-configuration, and
one stretch of at least three, in particular of five, consecutive deoxythymidines is located at the 3′ end of the oligodeoxyribonucleotide.

In one embodiment, the oligodeoxyribonucleotide comprises at least three CG dinucleotides.

In one embodiment, the oligodeoxyribonucleotide is single-stranded and/or partially or completely double-stranded.

In one embodiment, the oligodeoxyribonucleotide comprises two single-stranded loops and forms the shape of a dumbbell.

In one embodiment, all nucleotides are in D-configuration.

TLR-9 agonists, which are preferred according to the present invention, are in particular oligodeoxyribonucleotides as disclosed in EP 1 196 178, most preferred the immunomodulatory oligodeoxyribonucleotide known to the skilled person under the INN Lefitolimod (CAS registry number 1548439-51-5; also known as “MGN1703”; cf. FIG. 1; SEQ ID NO: 3). Lefitolimod (MGN1703) is a covalently-closed dumbbell-like immune surveillance reactivator with broad immunomodulatory effects on the innate and adaptive immune system. Lefitolimod is currently evaluated in a phase 3 trial in mCRC patients (IMPALA study), a phase 2 trial in SCLC patients (IMPULSE study) and in two phase ½ trials, (i) in solid tumors in combination with the checkpoint inhibitor ipilimumab and (ii) in HIV patients (TEACH study).

In the TLR-9 agonist of the present invention, the deoxyribose moieties of the oligodeoxyribonucleotide are linked by phosphodiester bonds. That means that the deoxyribose moieties of the oligodeoxyribonucleotide are not linked by phosphorothioate bonds.

Further preferred TLR-9 agonists suitable for use according to the present invention are disclosed in EP 2 655 623 B1 and EP 2 999 787 B1. Yet other preferred TLR-9 agonists according to the present invention are the TLR-9 agonists which are disclosed as SEQ ID NOs: 4 and 5 and 6 to 14 as shown in FIG. 2. However, the most preferred TLR-9 agonist for use in the present invention is Lefitolimod (SEQ ID NO: 3). Among the TLR-9 agonists comprising at least one nucleotide in L-configuration the sequences of SEQ ID NOs: 5, 8, 9 and 10 are preferred. SEQ ID NO: 5 is most preferred.

A tumor disease according to the present invention refers to a neoplasm which in turn refers to a type of abnormal and excessive growth of tissue. A neoplasm comprises benign and malignant neoplasms. A malignant neoplasm or tumor may also be referred to as cancer. A solid tumor can be referred to as an abnormal mass of tissue that usually does not contain cysts or liquid areas. Solid tumors may be benign or malignant. Examples of solid tumors are sarcomas, carcinomas, and lymphomas, such as mammary carcinomas, melanoma, skin neoplasms, gastrointestinal tumors, including colon carcinomas, stomach carcinomas, pancreas carcinomas, colon cancer, small intestine cancer, ovarial carcinomas, cervical carcinomas, lung cancer, prostate cancer, kidney cell carcinomas and/or liver metastases. Leukemias generally do not form solid tumors. Colon cancer or colorectal cancer is a malignant tumor arising from the inner wall of the colon or rectum. Colon cancer is a solid tumor.

Preferably, the tumor disease of the present invention is a solid tumor, preferably colon cancer. Other preferred tumors are colorectal cancer and melanoma.

Cells, for example T cells or macrophages, can infiltrate into the tumor. Tumor infiltration shall be understood to mean migration of cells from outside the tumor into the tumor tissue, e.g. in response to chemokines.

When cells infiltrate into the tumor, the cells are subsequently present in the tumor. In one embodiment of the invention, the TLR-9 agonist stimulates the infiltration of macrophages, preferably M1 macrophages, into the tumor and/or the treatment with said TLR-9 agonist leads to an increased ratio of M1 macrophages to M2 macrophages within the tumor. “Within the tumor” in the sense of the present invention refers to cells in the tumor. An increased ratio within the tumor may arise by an increased infiltration of the respective cells into the tumor or a conversion of, for example, M2 macrophages into M1 macrophages or an increased differentiation of macrophage precursors into M1 macrophages over M2 macrophages.

The “invasive margin” or “periphery” of a tumor shall mean a region centered on the border separating the malignant cell nests from the host tissue. The “center” of a tumor represents the remaining tumor area.

In the sense of the present invention, the “stimulation” of cell infiltration refers to the increase of the amount of the respective cell type within the tumor.

The term frequency refers to the frequency of a certain cell population within a parent cell population, for example the frequency of cytotoxic effector T cells (CD8+CD69+ Granzyme (Grz) B+) within the CD8+ T cell population.

The term ratio refers to the relation or ratio between two defined cell populations which do not overlap, for example the ratio of M1 macrophages to M2 macrophages.

The infiltration of the CD3+ T cells into the tumor can be identified by detection of CD3. CD3 has been used to detect lymphocytes in tumor samples (“Immunotherapy Doubts Fading” in GI Cancers, April 2016). Infiltration of macrophages can be detected by the markers CD11b and F4/80. It can then be discriminated between M1 macrophages (MHC-II/CD86 high) and M2 macrophages (MHC-II/CD86 low).

Tumor immune infiltration can also be determined using gene expression methods like microarray or RNA sequencing. Detection of gene expression specific for different kind of immune cell populations can then be used to determine the degree of lymphocyte or macrophage infiltration as has been shown in breast cancer for lymphocyte infiltration (Bedognetti, Davide; Hendrickx, Wouter; Marincola, Francesco M.; Miller, Lance D. “Prognostic and predictive immune gene signatures in breast cancer”. Current Opinion in Oncology. 27 (6): 433-444.) An active immune environment within the tumor often indicates a better prognosis as can be determined by the immunological constant of rejection.

As outlined above, T cells can be identified by the marker CD3. T cells are divided into two major populations: CD4+T helper cells and CD8+ cytotoxic T cells. CD4+T helper cells are essential for an activation of B cells to differentiate into antibody secreting plasma cells or memory B cells. CD4+ positive T cells also provide help for the differentiation of CD8+ T cells and secrete cytokines (Th1 cytokines—e.g. IFN-gamma or Th2 cytokines—e.g. IL-4) in order to shape an immune response. CD8+ T cells recognize specific peptides presented in the context of MHC I by tumor cells (or infected cells) and induce their destruction by soluble (perforin, granzymes, granulosin) or membrane bound (Fas-ligand) molecules. Cytotoxic effector T cells, i.e. activated and with cytolytic potential, can be detected by the markers CD8, CD69 and Granzyme B. Regulatory T cells can be detected by the markers CD3, CD4 and FOXP3.

As an alternative marker for CD3, Thy1 can be used, because CD3 can be downregulated upon activation of the cells.

Various methods are known to the skilled person to identify CD3+ T cells, they can be identified for example by flow cytometry or immunohistology (IHC). Whereas flow cytometry allows an in-depth analysis of cells using several markers in parallel, immunohistological analyses provide information regarding the localization of cells (tumor center/tumor periphery/surrounding healthy tissue). Hence using IHC the skilled person can identify the localization of the T-cells in the periphery or in the center of the tumor. The periphery or invasive marginmeans the border of tumor, adjacent to healthy tissue. It is preferred that the T-cells infiltrate the center of the tumor.

The oligodeoxyribonucleotide as used according to the invention comprises preferably a double-stranded stem and two single-stranded loops and forms the shape of a dumbbell. The oligodeoxyribonucleotide preferably is covalently closed. It is advantegous if least one CG dinucleotide is located in one or each of the single-stranded loops.

A “stem” according to the present disclosure shall be understood as a DNA double strand formed by base pairing either within the same DNA molecule (which is then partially self-complementary) or within different DNA molecules (which are partially or completely complementary). Intramolecular base pairing designates base pairing within the same molecules, and base pairing between different DNA molecules is termed as intermolecular base-pairing.

A “loop” within the meaning of the present disclosure shall be understood as an unpaired, single-stranded region either within or at the end of a stem structure.

A “dumbbell-shape” describes a oligonucleotide which comprises a double-stranded stem (base pairing within the same oligonucleotide) and two single stranded loops at both ends of the double-stranded stem.

In these dumbbell-shaped oligonucleotides preferably all nucleotides are in D-configuration. Furthermore it is preferred that the oligodeoxyribonucleotide comprises at least 50, preferably at least 80, most preferably 116 nucleotides. The nucleotide sequences of two preferred representatives of this type of TLR-9 agonists are depicted in FIG. 2 (SEQ ID NO: 3 and SEQ ID NO: 4).

The most preferred preferred TLR-agonist according to the invention is Lefitolimod (SEQ ID NO: 3).

L-DNA or nucleotides in L-configuration refer to nucleotides, which comprise L-deoxyribose as the sugar residue instead of the naturally occurring D-deoxyribose. L-deoxyribose is the enantiomer (mirror-image) of D-deoxyribose. DNA constructs partially or completely consisting of nucleotides in L-configuration can be partially or completely single- or double-stranded. L-DNA is equally soluble and selective as D-DNA. Yet, L-DNA is resistant towards degradation by naturally occurring enzymes, especially exonucleases, so L-DNA is protected against biological degradation. Therefore, L-DNA is very widely applicable.

Nucleotide sequences of preferred TLR-9 agonists comprising at least one nucleotide in L-configuration are depicted in FIG. 2 (SEQ ID NO: 5-14). Nucleotide sequences of SEQ ID NO: 5, 8, 9 and 10 are more preferred.

The most preferred TLR9-agonist comprising at least one nucleotide in L-configuration is the TLR9-agonist with a nucleotide sequence of SEQ ID NO: 5.

In a further embodiment of the invention a pharmaceutical composition comprising a TLR-9 agonist, preferably a TLR-9 agonist with all nucleotides in D-configuration, is provided, which is suitable for the treatment of a tumor disease, preferably colon cancer. This pharmaceutical composition comprises 1 mg/ml to 50 mg/ml, preferably 10 mg/ml to 20 mg/ml, more preferably 15 mg/ml of the TLR-9 agonist in phosphate-buffered saline (PBS), wherein the PBS has a pH of pH 6 to 8, in particular 7.0 to 7.5, and comprises
6 mg/ml to 12 mg/ml, preferably 8.8 mg/ml of sodium chloride,
0.1 mg/ml to 0.3 mg/ml, preferably 0.22 mg/ml of potassium chloride
0.1 mg/ml to 0.3 mg/ml, preferably 0.22 mg/ml of potassium dihydrogen phosphate and
1.0 mg/ml to 1.5 mg/ml, preferably 1.265 mg/ml of disodium hydrogen phosphate.

In a particularly preferred embodiment, the PBS has a pH of 7.2 to 7.6 and comprises
8.0 mg/ml of sodium chloride,
0.2 mg/ml of potassium chloride
0.2 mg/ml of potassium dihydrogen phosphate and
1.15 mg/ml of disodium hydrogen phosphate.

For the intratumoral injection, the preferred weekly dose in a human patient is about 10 to about 30 mg/week, preferably about 15 mg/week. For the subcutaneous injection, the preferred weekly dose in a human patient is about 100 to about 150 mg/week, preferably about 120 mg/week, which may be applied in several doses, for example about 60 mg twice a week or about 120 mg once a week.

In a preferred pharmaceutical composition of above, the TLR-9 agonist is Lefitolimod (SEQ ID NO: 3).

In a further embodiment, a pharmaceutical composition comprising a TLR-9 agonist comprising at least one nucleotide in L-configuration, is provided, which is suitable for the treatment of a tumor disease, preferably colon cancer. This pharmaceutical composition comprises 1 mg/ml to 30 mg/ml, preferably 10 mg/ml to 20 mg/ml, of the TLR-9 agonist comprising at least one nucleotide in L-configuration in glucose in a salt solution. Glucose is preferably in a concentration of 3% to 7%, even more preferably in a concentration of 5%. The salt solution is preferably 0.1 to 10 mM KCl, more preferably 0.5 to 3 mM KCl, even more preferably 1 mM KCl.

Thus, a preferred pharmaceutical composition comprising a TLR-9 agonist comprising at least one nucleotide in L-configuration comprises 10 mg/ml to 20 mg/ml of the TLR-9 agonist comprising at least one nucleotide in L-configuration in 5% glucose and 1 mM KCl.

A preferred TLR-9 agonist comprising at least one nucleotide in L-configuration is the oligodeoxynucleotide with the sequence of SEQ ID NO: 5.

According to one preferred embodiment of the invention the TLR-9 agonist, preferably Lefitolimod, is used as a monotherapy. The term “monotherapy” refers to the use of a drug as the single active pharmaceutical ingredient (API) to treat a particular disorder or disease. Thus, in a monotherapy the drug is administered without the combination with another drug.

In another preferred embodiment, a combination is provided which comprises a TLR-9 agonist and a chemotherapeutic and/or a checkpoint inhibitor for use in the treatment of a tumor disease, preferably of colon cancer, in a subject in need thereof, said TLR-9 agonist comprises
i. an oligodeoxyribonucleotide comprising at least one unmethylated CG dinucleotide, wherein C is deoxycytidine and G is deoxyguanosine and
ii. at least one stretch of at least three, in particular of four, consecutive deoxyguanosines,

and the deoxyribose moieties of the oligodeoxyribonucleotide are linked by phosphodiester bonds, wherein the treatment with said TLR-9 agonist stimulates the infiltration of CD3+ T cells into the tumor and/or stimulates the infiltration of macrophages, preferably M1 macrophages, into the tumor and/or leads to an increased ratio of M1 macrophages to M2 macrophages within the tumor.

The components of the combination, i. e. the TLR-9 agonist and the chemotherapeutic and/or the checkpoint inhibitor may thus be applied together at the same time and/or successively. Hence, in one embodiment, the subject to be treated has previously received and/or subsequently receives another cancer treatment. “Another cancer treatment” may for example refer to radiotherapy and/or application of a chemotherapeutic and/or a checkpoint inhibitor, preferably application of a chemotherapeutic and/or a checkpoint inhibitor.

The person skilled in the art is aware of suitable chemotherapeutics. Preclinical and clinical data have suggested that certain conventional chemotherapies may act in part through immune-stimulatory mechanisms. Such chemotherapeutics are preferred. For example, anthracyclines drive a regulated, immunogenic cell death (ICD) phenotype and directly block immunosuppressive pathways in the TME. Chemotherapeutic drugs, either inducing immunogenic cell death or blocking immunosuppressive pathways are e.g. oxaliplatin, carboplatin, cyclophosphamide, paclitaxel, 5-fluorouracil, doxorubicin, epirubucin, idarubicin, mitoxantrone, bleomycin, and bortezomib.

Instead or in addition to chemotherapeutics, “targeted anti-cancer agents”, such as tyrosine kinase inhibitors, may be used.

Radiotherapy is able to induce an immunogenic cell death, leading to the release of tumour antigens from dying cells that in turn promotes the activation of effector T-cells and boosts antitumor immune response, even against nonirradiated localizations (abscopal effect).

A checkpoint inhibitor blocks certain proteins expressed by some immune cells, such as T cells, and some cancer cells. These so-called checkpoint proteins help keep immune responses in check and can keep T cells from killing cancer cells. When these proteins are blocked, the “brakes” on the immune system are released and T cells are able to kill cancer cells. A checkpoint inhibitor is for example an inhibitor of PD-1, PD-L1, CTLA-4, TIM-3 or LAG3. Furthermore, so called co-stimulatory molecules like OX-40, CD137 (4-1BB) or GITR can be activated to enhance the immune response in order to optimize T cell activation. The person skilled in the art is aware of useful checkpoint inhibitors. Preferred checkpoint inhibitors are inhibitors of PD-1 such as Pembrolizumab and Nivolumab and inhibitors of PD-L1 such as Atezolizumab, Avelumab and Durvalumab. A preferred inhibitor of CTLA-4 is Ipilimumab. Other antibodies may also be used.

Chemotherapy or radiotherapy before application of a TLR-9 agonist may thus be advantageous because the chemotherapy or radiotherapy leads to a reduced tumor size and a release of tumor-associated antigens. The TLR-9 agonist may then activate antigen-presenting cells which in turn present the released tumor-associated antigens to T cells and stimulate the T cells.

Application of a TLR-9 agonist before a checkpoint inhibitor may be advantageous because application of the TLR-9 agonist leads to T cell activation which can in turn infiltrate into the tumor. The checkpoint inhibitor may then block inhibitory molecules on the already activated T cells in the tumor. T cells can thereby be fully activated and exert their anti-tumor effect.

Application of a checkpoint inhibitor before application of a TLR-9 agonist may be advantageous because the TLR-9 agonist may then abrogate a potential unresponsiveness to the checkpoint inhibitor. In other words, in case the tumor does not respond well to the checkpoint inhibitor, potentially because it is a cold tumor, the TLR-9 agonist may then modulate the tumor microenvironment in such a way that the responsiveness to the checkpoint inhibitor is restored. For example, the tumor may be resistant to treatment with a checkpoint inhibitor due to inhibitory molecules upregulated on the T cells. The de novo activated T cells activated by the TLR-9 agonist may not express these inhibitory molecules and may therefore be sensitive to a therapy with a checkpoint inhibitor.

In one embodiment, a composition is provided which comprises a TLR-9 agonist and a chemotherapeutic and/or a checkpoint inhibitor for use in the treatment of a tumor disease, preferably of colon cancer, in a subject in need thereof, said TLR-9 agonist comprises
i. an oligodeoxyribonucleotide comprising at least one unmethylated CG dinucleotide, wherein C is deoxycytidine and G is deoxyguanosine and
ii. at least one stretch of at least three, in particular of four, consecutive deoxyguanosines,

and the deoxyribose moieties of the oligodeoxyribonucleotide are linked by phosphodiester bonds, wherein the treatment with said TLR-9 agonist stimulates the infiltration of CD3+ T cells into the tumor and/or stimulates the infiltration of macrophages, preferably M1 macrophages, into the tumor and/or leads to an increased ratio of M1 macrophages to M2 macrophages within the tumor.

### BRIEF DESCRIPTION OF THE DRAWINGS

FIG. 1 Sequence and structure of Lefitolimod (MGN1703)

FIG. 2 Partial and complete sequences of the dumbbell-shaped TLR-9 agonists Lefitolimod (MGN1703), dSLIM 2006 (Partial sequences are used for the synthesis of the complete sequences), and sequences of TLR-9 agonists comprising L-nucleotides. The bold and underlined nucleotides are in L-configuration.

FIG. 3, FIG. 4, FIG. 5
Influence of Lefitolimod treatment on tumor growth; FIGS. 4 and 5 represent the single values of the values represented in FIG. 3.

FIG. 6 Quantification of CD3+ T cells within the center of tumors after treatment with Lefitolimod

FIG. 7 Quantification of CD8+ T cells within the center of tumors after treatment with Lefitolimod

FIG. 8 and FIG. 9
Correlation of infiltrated CD8+ T cells with the reduction of tumor growth

FIG. 10, FIG. 11 and FIG. 12
Influence of a TLR-9 agonist comprising L-nucleotides (SEQ ID NO: 5) treatment on tumor growth after treatment with Lefitolimod; FIGS. 11 and 12 represent the single values of the values represented in FIG. 10.

FIG. 13 Quantification of CD3+ T cells within the center of tumors after treatment with a TLR-9 agonist comprising L-nucleotides (SEQ ID NO: 5)

FIG. 14 Quantification of CD8+ T cells within the center of tumors after treatment with a TLR-9 agonist comprising L-nucleotides (SEQ ID NO: 5)

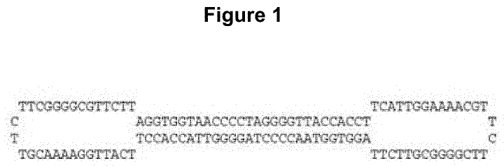

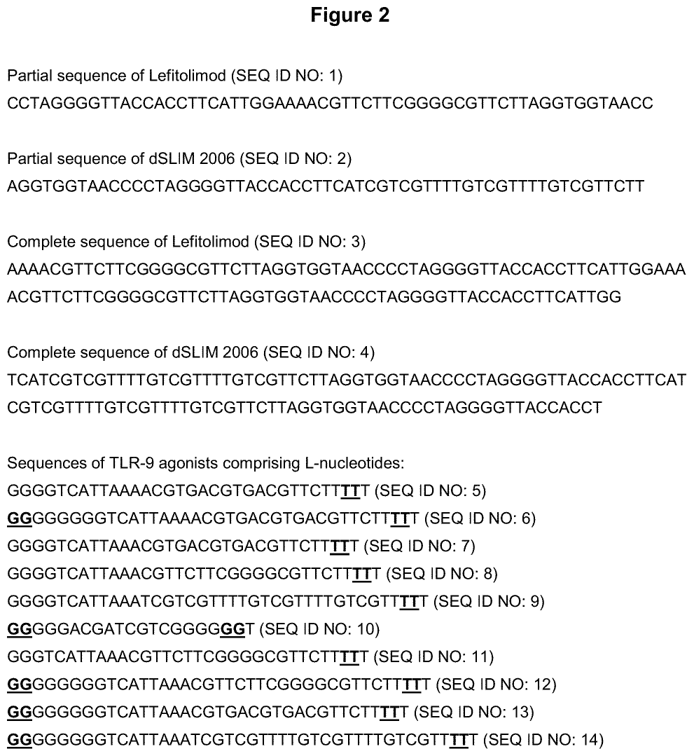

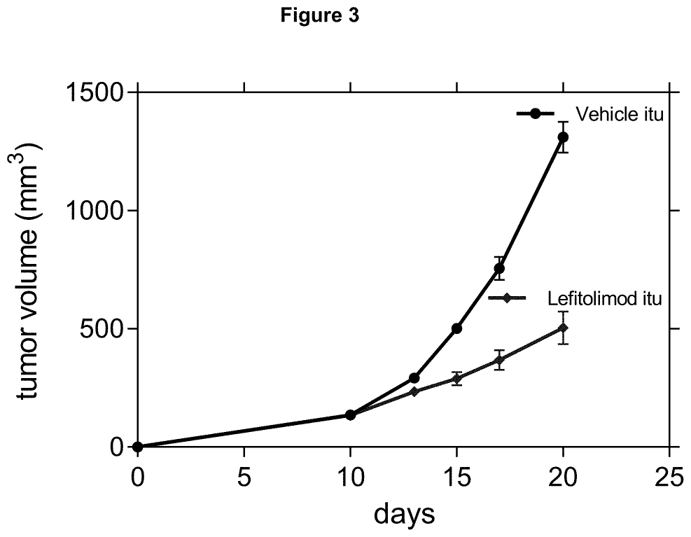

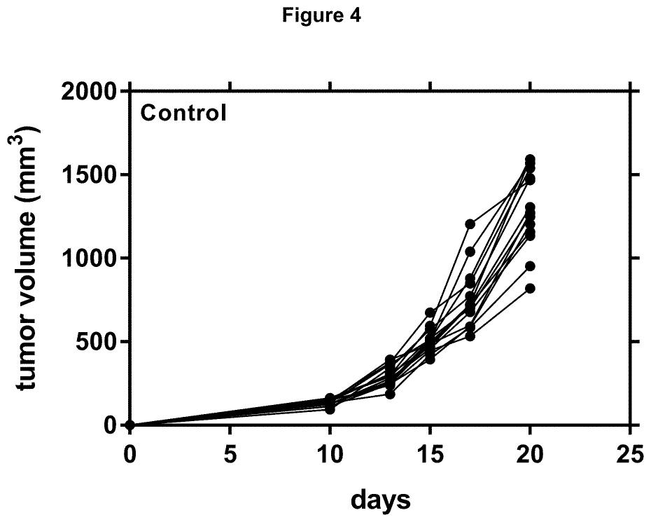

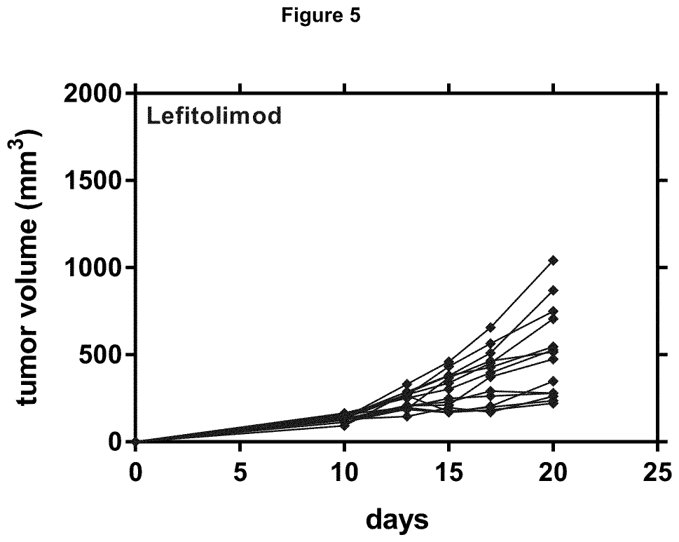

FIG. 15 and FIG. 16
Correlation of infiltrated CD8+ T cells with the reduction of tumor growth after treatment with a TLR-9 agonist comprising L-nucleotides (SEQ ID NO: 5)

FIG. 17
Analysis of T cell populations within tumors by flow cytometry after treatment with Lefitolimod

FIG. 18
Analysis of CD8+ T cells, cytotoxic effector T cells (CD8+CD69+GrzB+) and regulatory T cells by flow cytometry after treatment with Lefitolimod

FIG. 19
Analysis of macrophages within tumors by IHC after treatment with Lefitolimod

FIG. 20
Analysis of macrophages within tumors by flow cytometry after treatment with Lefitolimod

FIG. 21-23
Influence of subcutaneous Lefitolimod treatment on tumor growth; FIGS. 22 and 23 represent the single values of the values represented in FIG. 21.

FIG. 24
Quantification of CD8+ T cells within the center of tumors after subcutaneous treatment with Lefitolimod

FIG. 25
Quantification of CD8+ T cells within the tumor margin after subcutaneous treatment with Lefitolimod

FIG. 26
Comparison of secretion of IFN-alpha and IP-10 from human PBMC and HLA-DR expression on pDC after treatment with different TLR-9 agonists comprising L-nucleotides (SEQ ID NO: 5-11)

FIG. 27
Comparison of secretion of IFN-alpha and IP-10 from human PBMC after treatment with different TLR-9 agonists comprising L-nucleotides (SEQ ID NO: 7-9 and 12-14)

### SPECIFIC EMBODIMENTS

1. A TLR-9 agonist for use in the treatment of a tumor disease, preferably of colon cancer, in a subject in need thereof, said TLR-9 agonist comprises
   i. an oligodeoxyribonucleotide comprising at least one unmethylated CG dinucleotide, wherein C is deoxycytidine and G is deoxyguanosine and
   ii. at least one stretch of at least three, in particular of four, consecutive deoxyguanosines,

and the deoxyribose moieties of the oligodeoxyribonucleotide are linked by phosphodiester bonds, wherein the treatment with said TLR-9 agonist stimulates the infiltration of CD3+ T cells into the tumor.

In another embodiment 1, the treatment with said TLR-9 agonist stimulates the infiltration of macrophages, preferably M1 macrophages, into the tumor and/or the treatment with said TLR-9 agonist leads to an increased ratio of M1 macrophages to M2 macrophages within the tumor.
2. The TLR-9 agonist for use according to embodiment 1, wherein the infiltration of the tumor by the CD3+ T cells and/or by the macrophages, preferably M1 macrophages, is stimulated compared with the infiltration without the treatment of the TLR-9 agonist.

In another embodiment 2, the ratio of M1 macrophages to M2 macrophages within the tumor is increased compared to the ratio of M1 macrophages to M2 macrophages without the treatment of the TLR-9 agonist.
3. The TLR-9 agonist for use according to embodiment 1 or 2, wherein the CD3+ T cells are CD4+ or CD8+ T cells, preferably CD8+ T cells.

In another embodiment 3, the treatment with said TLR-9 agonist leads to an increased frequency of cytotoxic effector T cells (CD8+CD69+ Granzyme B+) within the CD8+ T cell population in the tumor.

In another embodiment 3, the treatment with said TLR-9 agonist leads to an increased ratio of CD8+ T cells, preferably of cytotoxic effector T cells (CD8+CD69+ Granzyme B+), to regulatory T cells.
4. The TLR-9 agonist for use according to any of the preceding embodiments, wherein the tumor is infiltrated in its periphery and/or its center, preferably in its center.
5. The TLR-9 agonist for use according to any of the preceding embodiments, wherein the tumor is a solid tumor, preferably colon cancer, and the subject to be treated is a human.
6. The TLR-9 agonist for use according to any of the preceding embodiments, wherein the TLR-9 agonist is administered intratumorally or subcutaneously.
7. The TLR-9 agonist for use according to any of the preceding embodiments, wherein the at least one CG dinucleotide is part of a sequence N¹N²CGN³N⁴, wherein N¹N² is AA, TT, GG, GT, GA or AT and N³N⁴ is CT, TT, TG or GG and C is deoxycytidine, G is deoxyguanosine, A is deoxyadenosine, and T is deoxythymidine.
8. The TLR-9 agonist for use according to any of the preceding embodiments, wherein the oligodeoxyribonucleotide comprises at least one nucleotide in L-configuration, preferably the at least one nucleotide in L-configuration is comprised within the terminal five nucleotides of at least one end of the oligodeoxyribonucleotide, preferably within the terminal five nucleotides of the 3′ end of the oligodeoxyribonucleotide.

In one embodiment 8, the TLR-9 agonist comprising at least one nucleotide in L-configuration has one or more of the following features:
the at least one stretch of at least three, in particular of four, consecutive deoxyguanosines is located at the 5′ end of the oligodeoxyribonucleotide,
at least one stretch of at least three consecutive deoxyguanosines is located between two CG dinucleotides,
one stretch of at least three, in particular of five, consecutive deoxythymidines is located at the 3′ end of the oligodeoxyribonucleotide,
at least one of the three 3′ terminal and/or 5′ terminal deoxynucleotides is in L-configuration,
two of the three 3′ terminal deoxynucleotides are in L-configuration,
the first and the second 5′ terminal deoxynucleotide are in L-configuration.

In a particularly preferred embodiment 8, in the TLR-9 agonist comprising at least one nucleotide in L-configuration
the at least one stretch of at least three, in particular of four, consecutive deoxyguanosines is located at the 5′ end of the oligodeoxyribonucleotide, and
two of the three 3′ terminal deoxynucleotides are in L-configuration, and
one stretch of at least three, in particular of five, consecutive deoxythymidines is located at the 3′ end of the oligodeoxyribonucleotide.
9. The TLR-9 agonist for use according to any of the preceding embodiments, wherein the oligodeoxyribonucleotide comprises at least three CG dinucleotides.
10. The TLR-9 agonist for use according to any of the preceding embodiments, wherein the oligodeoxyribonucleotide is single-stranded and/or partially or completely double-stranded.
11. The TLR-9 agonist for use according to any of the preceding embodiments, wherein the oligodeoxyribonucleotide comprises two single-stranded loops and forms the shape of a dumbbell.
12. The TLR-9 agonist for use according to embodiment 11, wherein all nucleotides are in D-configuration.
13. The TLR-9 agonist for use according to embodiments 11 or 12 having the sequence of SEQ ID NO: 3.
14. The TLR-9 agonist for use according to embodiment 8 having the sequence of SEQ ID NO: 5.
15. A method for increasing the infiltration of CD3+ T cells, preferably CD8+ T cells, into a tumor, preferably colon cancer, comprising administering a TLR-9 agonist to a patient in need thereof, wherein the TLR-9 agonist comprises
   i. an oligodeoxyribonucleotide comprising at least one unmethylated CG dinucleotide, wherein C is deoxycytidine and G is deoxyguanosine and
   ii. at least one stretch of at least three, in particular of four, consecutive deoxyguanosines and

wherein the deoxyribose moieties of the oligodeoxyribonucleotide are linked by phosphodiester bonds.

In another embodiment 15, said method serves for increasing the frequency of cytotoxic effector T cells (CD8+CD69+ Granzyme B+) within the CD8+ T cell population in the tumor.

In another embodiment 15, said method serves for increasing the ratio of CD8+ T cells, preferably of cytotoxic effector T cells (CD8+CD69+ Granzyme B+), to regulatory T cells.

In another embodiment 15, said method serves for increasing the infiltration of macrophages, preferably M1 macrophages, into the tumor and/or for increasing a ratio of M1 macrophages to M2 macrophages within the tumor.
16. The TLR-9 agonist for use according to embodiment 8, wherein the oligodeoxyribonucleotide comprises at least 20 nucleotides, preferably 30 to 35 nucleotides.
17. The TLR-9 agonist for use according to embodiment 8 or 16, wherein the at least one stretch of at least three, in particular of four, consecutive deoxyguanosines is in D-configuration and is located within the terminal six nucleotides of at least one end of the oligodeoxyribonucleotide, preferably within the terminal six nucleotides of the 5′ end of the oligodeoxyribonucleotide.
18. The TLR-9 agonist for use according to embodiment 11, wherein at least three, in particular four, consecutive deoxyguanosines are located in between two CG dinucleotides.
19. The TLR-9 agonist for use according to embodiment 11, wherein at least five nucleotides are located between two CG dinucleotides, excluding deoxyguanosine.
20. The TLR-9 agonist for use according to any of the preceding embodiments, wherein the at least one CG dinucleotide is located in a single- and/or double-stranded region of the oligodeoxyribonucleotide.
21. The TLR-9 agonist for use according to any of embodiments 1 to 10, wherein the oligodeoxyribonucleotide comprises a double-stranded stem and at least one single-stranded loop.
22. The TLR-9 agonist for use according to embodiment 11 or 21, wherein the oligodeoxyribonucleotide comprises at least 50, preferably at least 80, most preferably 116 nucleotides.
23. The TLR-9 agonist for use according to embodiment 11 or 21, wherein the oligodeoxyribonucleotide comprises at most 200, preferably at most 150, most preferably 116 nucleotides.
24. The TLR-9 agonist for use according to embodiment 11 or 21, wherein the at least one CG dinucleotide is located in
   i. one or each of the single-stranded loops, preferably in each of the single-stranded loops or
   ii. the double-stranded stem, or
   iii. one or each of the single-stranded loops and in the double-stranded stem.
25. The TLR-9 agonist for use according to embodiment 11 or 21, wherein the oligodeoxyribonucleotide comprises a double-stranded stem and at least one single-stranded loop and the at least one CG dinucleotide is located in the single-stranded loop.
26. The TLR-9 agonist for use according to embodiment 25, wherein three CG dinucleotides are located in each of the single-stranded loops.
27. The TLR-9 agonist for use according to embodiment 11 or 21, wherein one or each of the single-stranded loops comprises at least 20 nucleotides, preferably consists of 30 nucleotides.
28. The TLR-9 agonist for use according to embodiment 27, wherein each of the single-stranded loops consists of 30 nucleotides and is of identical sequence.
29. The TLR-9 agonist for use according to embodiment 11 or 21, wherein the double-stranded stem comprises at least 15, preferably at least 20, most preferably consists of 28 base pairs.
30. The TLR-9 agonist for use according to embodiment 11 or 21, wherein the double-stranded stem comprises at most 90, preferably at most 60, most preferably consists of 28 base pairs.
31. The TLR-9 agonist for use according to embodiment 11, wherein each of the single-stranded loops consists of 30 nucleotides, the double-stranded stem consists of 28 base pairs and three CG dinucleotides are located in each of the single-stranded loops.
32. The TLR-9 agonist for use according to embodiment 11, wherein the TLR-9 agonist consists of twice a partially hybridized sequence of SEQ ID NO: 1.
33. The TLR-9 agonist for use according to embodiment 11, wherein the TLR-9 agonist consists of twice a partially hybridized sequence of SEQ ID NO: 2.
34. The TLR-9 agonist for use according to embodiment 11, wherein the TLR-9 agonist consists of the sequence of SEQ ID NO: 4.
35. The TLR-9 agonist for use according to any of the preceding embodiments, wherein at least one nucleotide is modified with a functional group selected from the group comprising carboxyl, amine, amide, aldimine, ketal, acetal, ester, ether, disulfide, thiol and aldehyde groups.
36. The TLR-9 agonist for use according to embodiment 35, wherein the modified nucleotide is linked to a compound selected from the group comprising peptides, proteins, carbohydrates, antibodies, lipids, micelles, vesicles, synthetic molecules, polymers, micro projectiles, metal particles, nanoparticles, and a solid phase.
37. The method according to embodiment 15, wherein the TLR-9 agonist is as defined in any of the preceding embodiments.
38. A pharmaceutical composition comprising 1 mg/ml to 50 mg/ml, preferably 10 mg/ml to 20 mg/ml, more preferably 15 mg/ml of a TLR-9 agonist, preferably a TLR-9 agonist with all nucleotides in D-configuration, in PBS for use in treating a tumor disease, preferably colon cancer, wherein the PBS has a pH of pH 6 to 8, in particular 7.2 to 7.6, and comprises
   6 mg/ml to 12 mg/ml, preferably 8.0 mg/ml of sodium chloride,
   0.1 mg/ml to 0.3 mg/ml, preferably 0.2 mg/ml of potassium chloride
   0.1 mg/ml to 0.3 mg/ml, preferably 0.2 mg/ml of potassium dihydrogen phosphate and
   1.0 mg/ml to 1.5 mg/ml, preferably 1.15 mg/ml of disodium hydrogen phosphate.

In a preferred embodiment 38, the TLR-9 agonist is Lefitolimod (SEQ ID NO: 3).

In another embodiment 38, a pharmaceutical composition comprises 1 mg/ml to 30 mg/ml, preferably 10 mg/ml to 20 mg/ml, of a TLR-9 agonist comprising at least one nucleotide in L-configuration in glucose in a salt solution. Glucose is preferably in a concentration of 3% to 7%, even more preferably in a concentration of 5%. The salt solution is preferably 0.1 to 10 mM KCl, more preferably 0.5 to 3 mM KCl, even more preferably 1 mM KCl.

Thus, a preferred pharmaceutical composition comprising a TLR-9 agonist comprising at least one nucleotide in L-configuration comprises 10 mg/ml to 20 mg/ml of the TLR-9 agonist comprising at least one nucleotide in L-configuration in 5 glucose and 1 mM KCl.

A preferred TLR-9 agonist comprising at least one nucleotide in L-configuration is the oligodeoxynucleotide with the sequence of SEQ ID NO: 5.
39. A method of treatment of a tumor disease, preferably colon cancer, in a subject comprising administering a TLR-9 agonist, wherein the TLR-9 agonist comprises
   i. an oligodeoxyribonucleotide comprising at least one unmethylated CG dinucleotide, wherein C is deoxycytidine and G is deoxyguanosine and
   ii. at least one stretch of at least three, in particular of four, consecutive deoxyguanosines,

and the deoxyribose moieties of the oligodeoxyribonucleotide are linked by phosphodiester bonds, wherein the treatment with said TLR-9 agonist stimulates the infiltration of CD3+ T cells into the tumor and/or stimulates the infiltration of macrophages, preferably M1 macrophages, into the tumor and/or leads to an increased ratio of M1 macrophages to M2 macrophages within the tumor.

Preferably, the TLR-9 agonist is Lefitolimod.
40. Combination comprising a TLR-9 agonist and a chemotherapeutic and/or a checkpoint inhibitor for use in the treatment of a tumor disease, preferably of colon cancer, in a subject in need thereof, said TLR-9 agonist comprises
   i. an oligodeoxyribonucleotide comprising at least one unmethylated CG dinucleotide, wherein C is deoxycytidine and G is deoxyguanosine and
   ii. at least one stretch of at least three, in particular of four, consecutive deoxyguanosines,

and the deoxyribose moieties of the oligodeoxyribonucleotide are linked by phosphodiester bonds, wherein the treatment with said TLR-9 agonist stimulates the infiltration of CD3+ T cells into the tumor and/or stimulates the infiltration of macrophages, preferably M1 macrophages, into the tumor and/or leads to an increased ratio of M1 macrophages to M2 macrophages within the tumor.

Preferably, the TLR-9 agonist is Lefitolimod.

### EXAMPLES

### Example 1: TLR-9 Agonists Modulate the Tumor Microenvironment

The benefits of the use of the TLR9 agonists as of the invention are exemplified by two TLR9 agonists, Lefitolimod (SEQ ID NO: 3) and a TLR-9 agonist comprising L-nucleotides (SEQ ID NO: 5). The potential of Lefitolimod and the TLR-9 agonist comprising L-nucleotides (SEQ ID NO: 5) to modulate the tumor microenvironment (TME) with regard to an infiltration of immune cells necessary for an antitumor immune response was evaluated. Both, Lefitolimod and the TLR-9 agonist comprising L-nucleotides (SEQ ID NO: 5), are particularly preferred embodiments of the invention.

A syngeneic murine colon carcinoma model (CT26) was used to demonstrate these effects. The biological modulation of the TME was evaluated using immunohistochemistry (IHC) and flow cytometry.

Material/Methods

1.) Immunohistochemistry (IHC) after Intratumoral Administration

Balb/c mice were subcutaneously inoculated in the flank at day 0 with 0.05×10⁶ CT26 tumor cells with matrigel. Mice were randomized by their tumor volumes with cage equalizer software into the treatment groups. The compounds (dissolved in PBS) or the vehicle (PBS) were administered by the intratumoral route at day 10 when tumors reached a volume of approximately 140 mm³. 200 μg Lefitolimod or 200 μg of the TLR-9 agonist comprising L-nucleotides (SEQ ID NO: 5) were applied 3 times per week. At day 21 mice were sacrificed and tumors were collected for IHC. For CD3 staining as well as staining of the macrophage marker F4/80 tissues were fixed in formalin and embedded in paraffin. For CD8 staining cryosections were performed. IHC staining was performed by standard methods. Staining for immune cells was analyzed for the tumor center as well as the invasive margin. CD3-staining was quantified by the Definiens software (Definiens AG, Munich, Germany) and CD8 staining was quantified manually by a scoring strategy:

Scoring strategy for CD8 from 1 to 4:

1=no labelling

2=few labelling

3=intermediate labelling

4=intense labelling

Scoring was performed by one IHC operator and checked by another IHC operator who did not participate in the study.

2.) Flow Cytometry after Intratumoral Administration

The in-life part (CT 26 mice) was described as above with the exception that 250 μg lefitolimod were used. At day 21 mice were sacrificed and tumors were collected for an analysis of tumor-infiltrating leucocytes by flow cytometry. Two panels of fluorophore-conjugated antibodies were used for an evaluation of T cells and macrophages:

| Panel I | Panel II |
|---|---|
| CD4 - PerCPVio700 | F4/80 - BV510 |
| CD8a - BV510 | CD45 - PeCy7 |
| CD45 - PeCy7 | CD11b - APC |
| Thy1 - APC-Cy7 | MHC II - PerCPCy5.5 |
| FoxP3 - PE | CD86 - PE |
| Granzyme B - Alexa647 | |
| CD69 - BV421 | |

The viability dye eFluor 520 was used to discriminate dead cells. CD45 is a marker for leukocytes and serves for discriminating leukocytes from tumor cells.

The following markers have been used for detecting the following cell types:

| Cell Type | Marker |
|---|---|
| CD3+ T cells | Thy1 |
| CD8+ T cells | Thy1+ CD8+ |
| Cytotoxic effector T cells | Thy1+ CD8+ CD69+ Granzyme B+ |
| CD4+ regulatory T cells | Thy1+ CD4+ FOXP3+ |
| M1 macrophages | CD11b+ F4/80 + MHC-II/CD86 high |
| M2 macrophages | CD11b+ F4/80 + MHC-II/CD86 low |

Thy1 was used instead of CD3, because CD3 can be downregulated upon activation of the cells.

3.) IHC after Subcutaneous Administration

Balb/c mice were subcutaneously inoculated in the right lower flank with 0.5×10⁶ CT26 tumor cells at day 0. Mice were randomized based on their body weight using StudyDirector™ software (Studylog Systems, Inc., South San Francisco, Calif., USA) into the treatment groups. Lefitolimod (dissolved in PBS) or the vehicle (PBS) was administered by distant subcutaneous route (left front flank nearby axillary lymph nodes) at day 2. 250 μg Lefitolimod was applied 3 times per week until day 16. At day 17 mice were sacrificed and tumors were collected for IHC. Tissues were fixed in formalin and embedded in paraffin. IHC staining for CD8 was performed by standard methods known to the person skilled in the art. All stained sections were scanned with NanoZoomer-HT 2.0 Image System (Hamamatsu photonics). Five representative fields of each sample were chosen for analysis. Quantification of scanned areas was performed with ImageJ software (https://imagej.net/). IHC scores are presented as the ratio of the average of the positive cell counts in the five fields against the total cell numbers in the fields. IHC scoring of the tumor margin and the tumor center was done separately.

Results

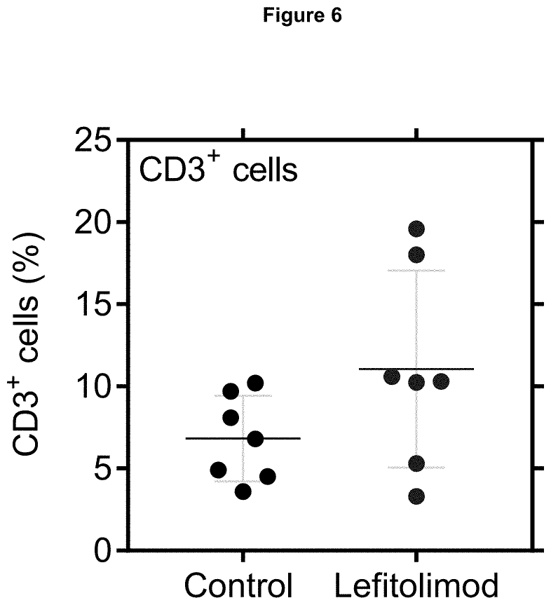

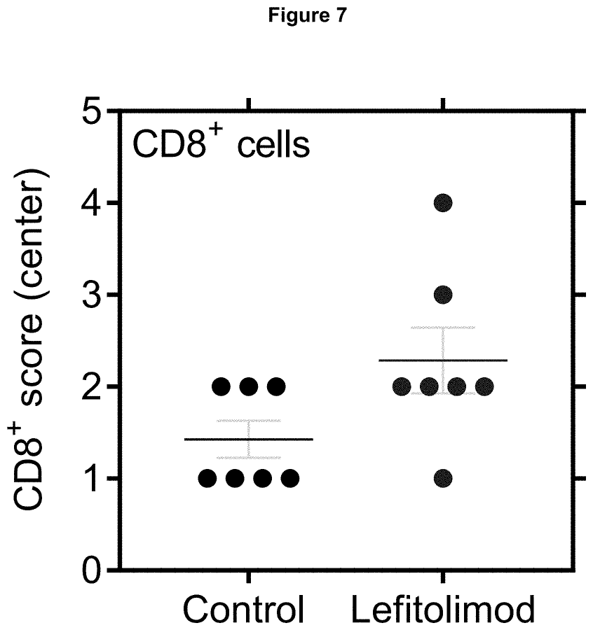

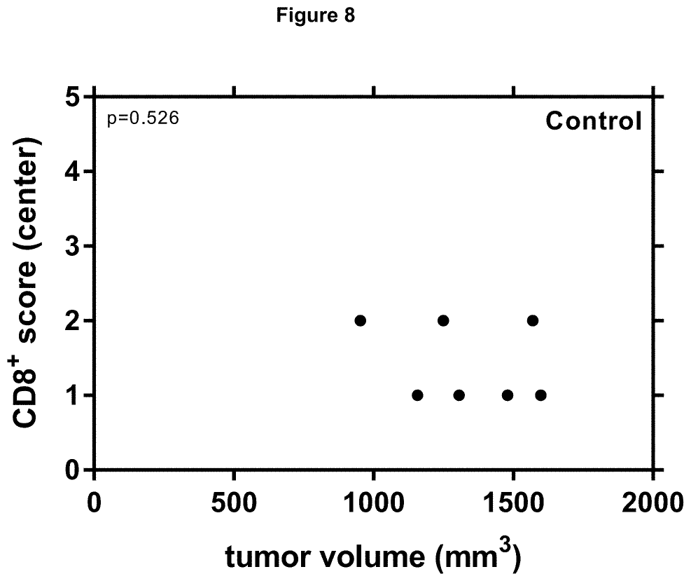

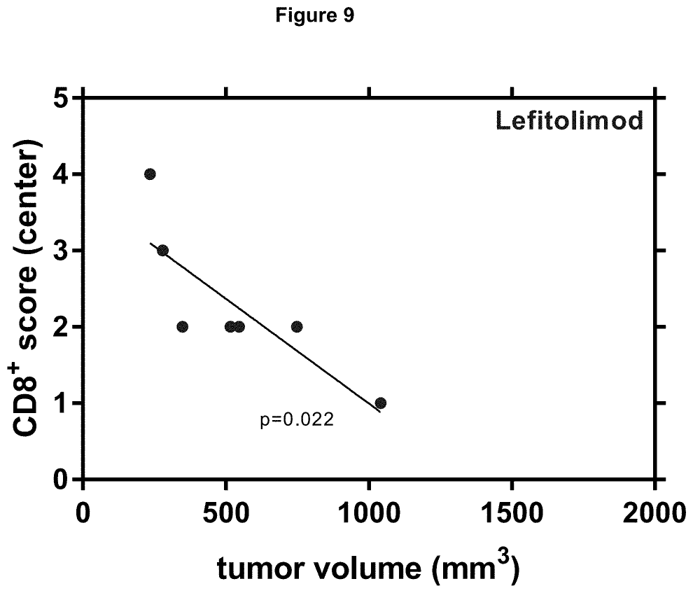

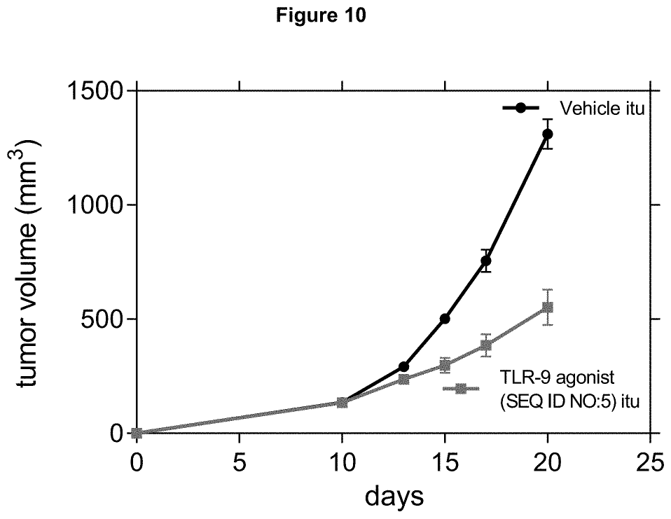

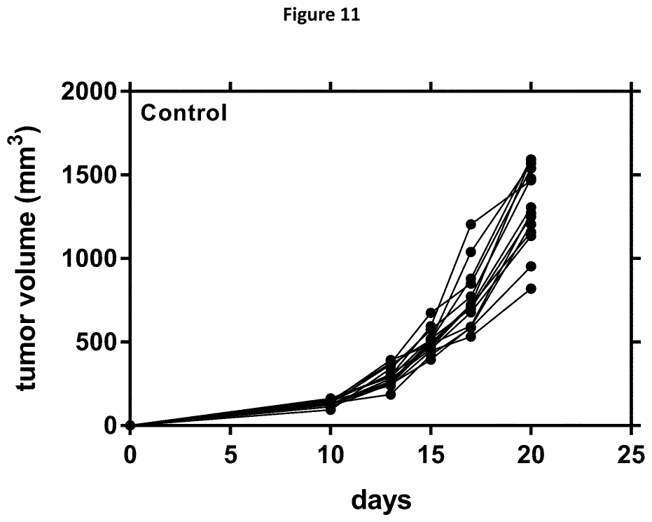

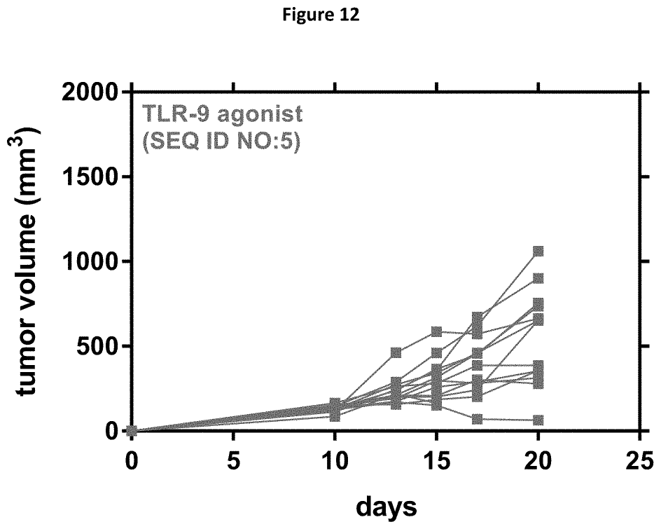

Treatment with Lefitolimod administered intratumorally resulted in a reduced tumor growth in comparison to the vehicle control (FIG. 3, FIG. 4 and FIG. 5). This was accompanied by an enhanced infiltration of CD3+ T cells into the tumor center (FIG. 6). Lefitolimod treatment also resulted in an infiltration of cytotoxic CD8+ T cells into the tumor center (FIG. 7). There was a correlation between CD8+ T cell infiltration and the reduction of tumor growth (p=0.022; FIG. 8 and FIG. 9).

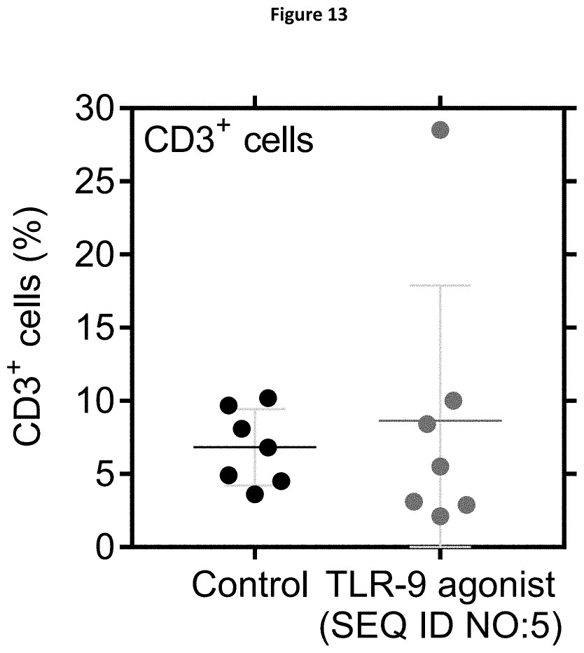

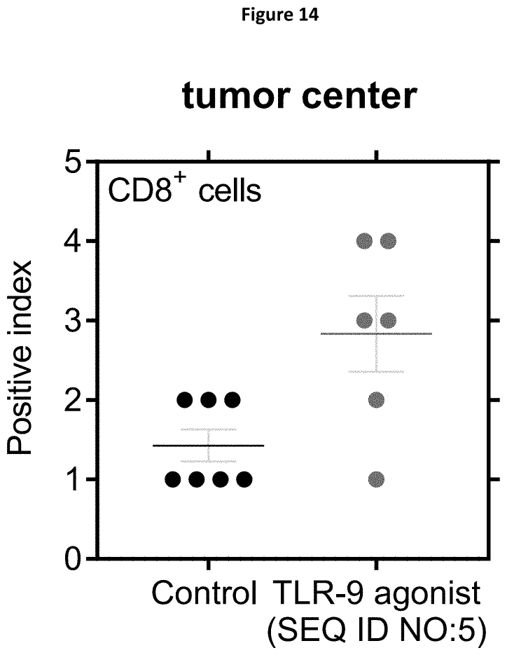

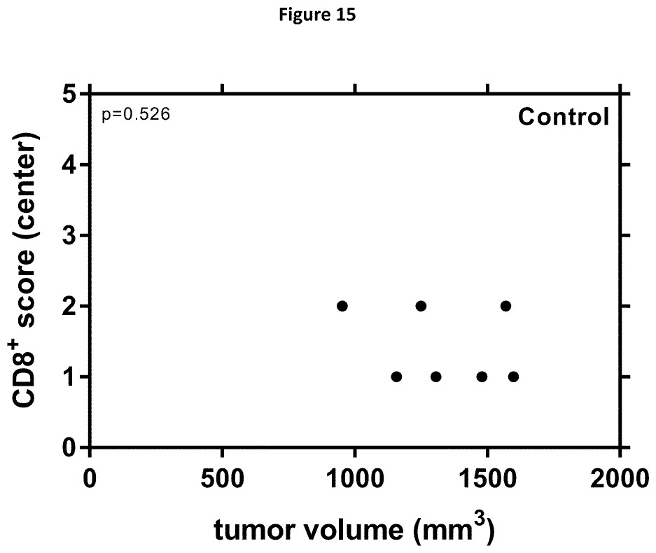

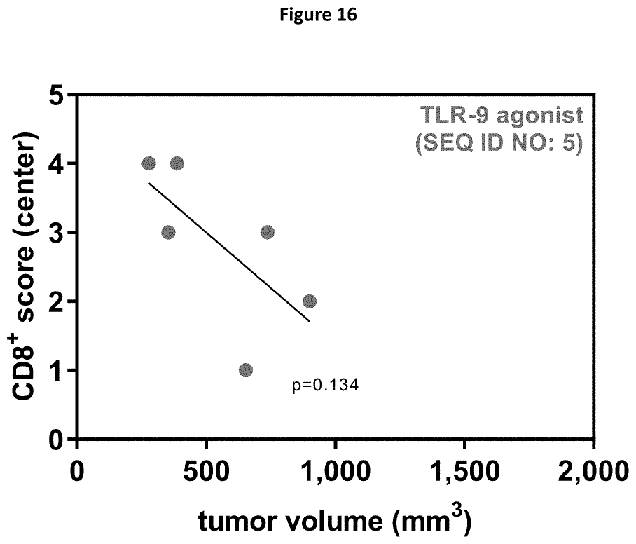

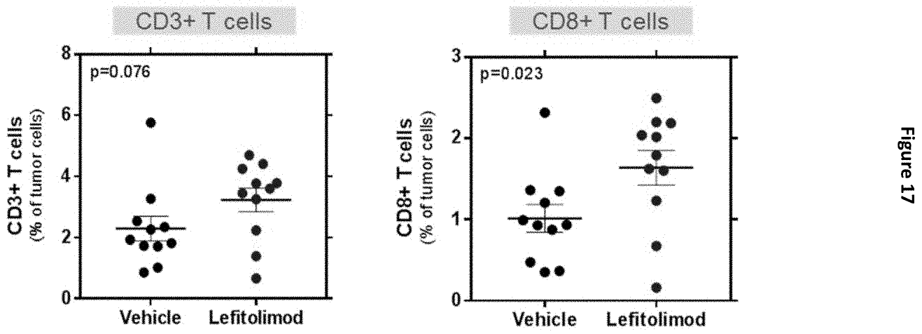

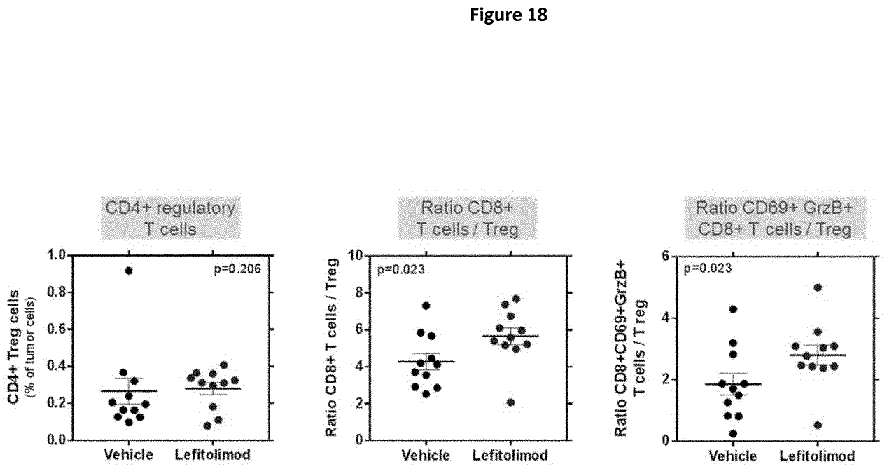

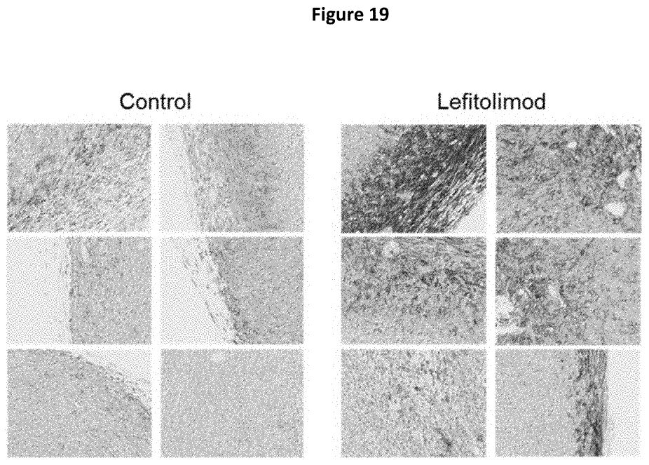

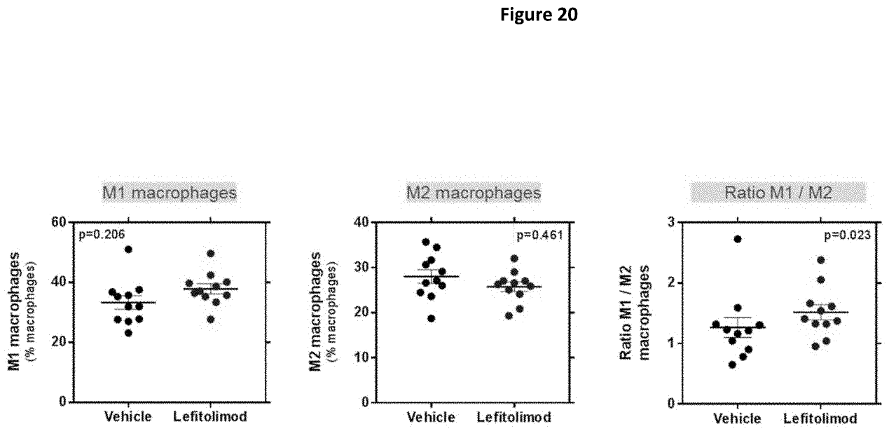

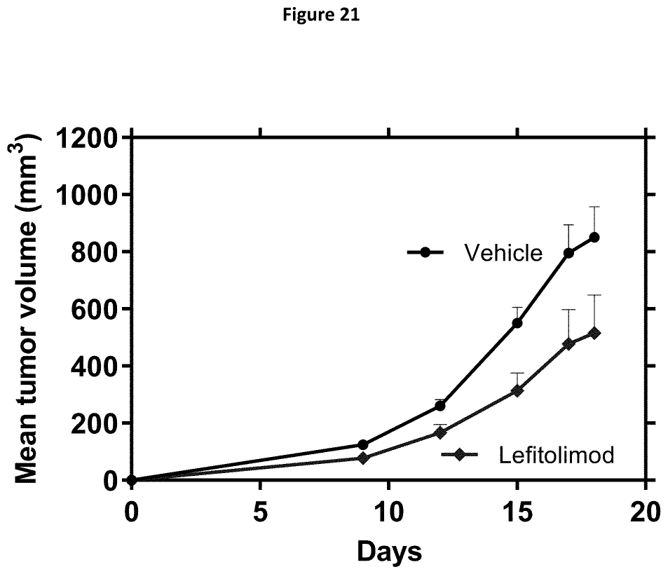

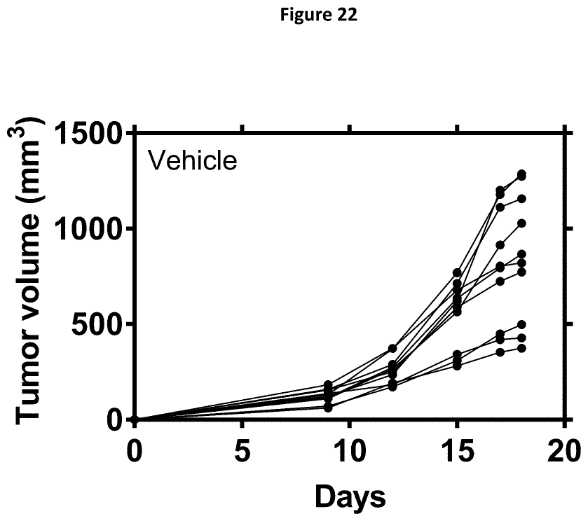

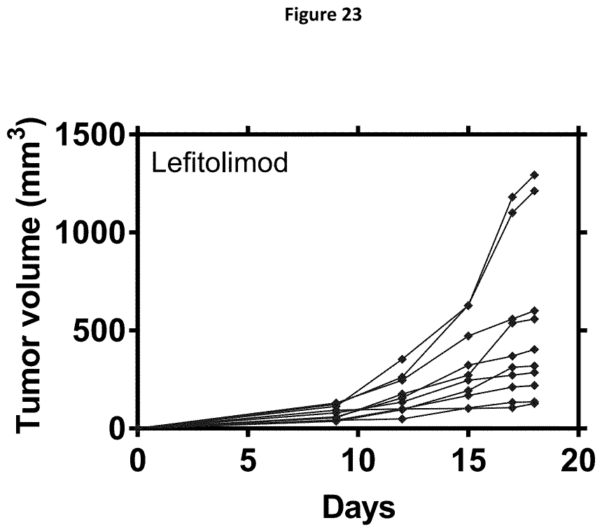

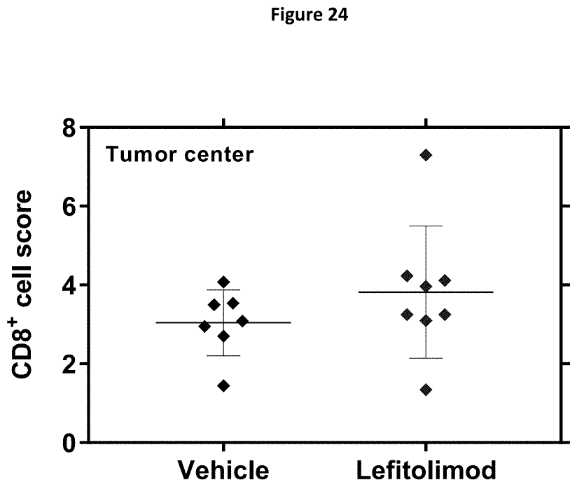

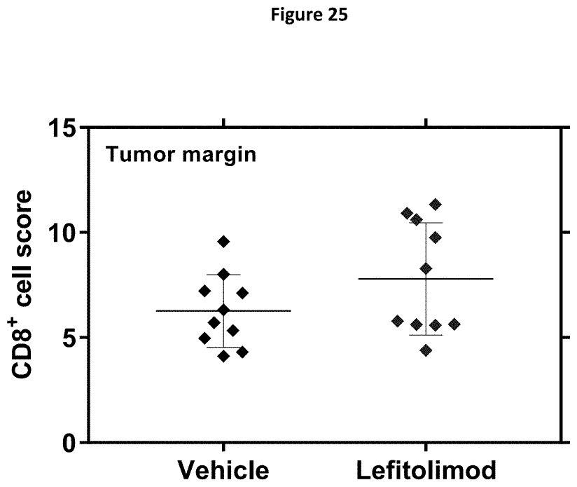

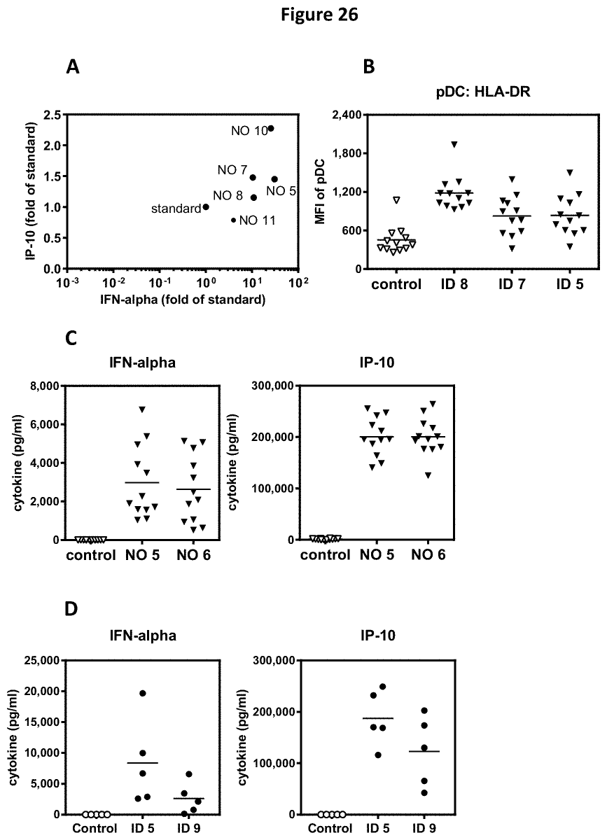

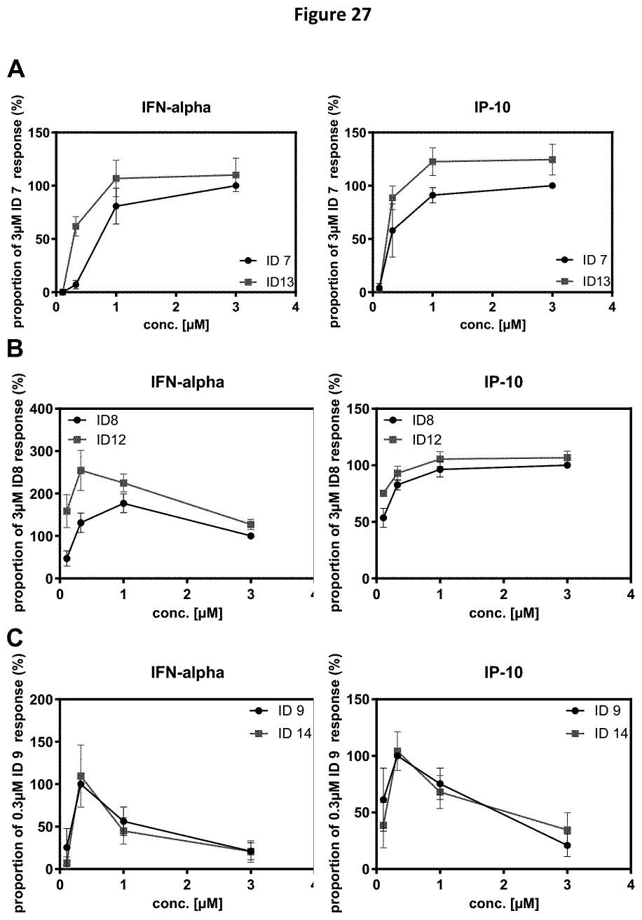

Treatment with the TLR-9 agonist comprising L-nucleotides (SEQ ID NO: 5) (administered intratumorally) resulted in a reduced tumor growth in comparison to the vehicle control (FIG. 10, FIG. 11 and FIG. 12). A moderate infiltration of CD3+ T cells into the tumor was observed (FIG. 13). Treatment with the TLR-9 agonist comprising L-nucleotides (SEQ ID NO: 5) also resulted in an infiltration of cytotoxic CD8+ T cells into the tumor center (FIG. 14). There was a trend towards a correlation between CD8+ T cell infiltration and the reduction of tumor growth (FIG. 15 and FIG. 16).

The infiltration of CD3+ T cells as well as CD8+ T cells by Lefitolimod (administered intratumorally) was confirmed by flow cytometry. An increase of both cell populations within the whole tumor cells was observed. (FIG. 17).

Furthermore, there was a significant increase in the ratio of anti-tumorigenic cytotoxic effector T cells (CD8+CD69+GrzB+) to pro-tumorigenic regulatory T cells by Lefitolimod (administered intratumorally). Cytotoxic effector T cells express cytolytic molecules, e.g. Granzyme B (Grz B), and are therefore capable of destroying tumor cells. The same was observed for the whole CD8+ T cell population. The frequency of regulatory T cells remained unchanged (FIG. 18).

Lefitolimod treatment (administered intratumorally) resulted in an attraction of macrophages into the tumor (FIG. 19).

Results from flow cytometric analyses showed an increase of M1 macrophages (CD86 and MHC II high), a decrease of M2 macrophages (CD86 and MHC II low) and an increased ratio of M1 macrophages to M2 macrophages within the tumor by Lefitolimod (administered intratumorally) (FIG. 20).

Statistical analyses of the data of FIG. 17, FIG. 18 and FIG. 20 have been done using a Kolmogorov-Smirnov test.

Tumor volume was not only reduced after intratumoral, but also after distant subcutaneous administration of Lefitolimod (FIGS. 21-23). Lefitolimod treatment (administered subcutaneously) also resulted in an infiltration of cytotoxic CD8+ T cells into the center and margin of tumors (FIG. 24-25).

Thus, a TLR-9 agonist according to the invention is beneficial for modulating the TME and for promoting an anti-tumor response in that a conversion of M2 macrophages into M1 macrophages is promoted and/or an infiltration of M1 macrophages and/or CD8+ T cells, e.g. cytotoxic effector T cells (CD8+CD69+GrzB+), into the tumor is stimulated.

### Example 2: TLR-9 Agonists Comprising at Least One Nucleotide in L-Configuration Activate the Immune System

Different TLR-9 agonists comprising at least one nucleotide in L-configuration were tested for their potential to activate the immune system, in particular to modulate the tumor microenvironment.

Methods

Buffy coats from anonymized healthy donors were obtained from the “DRK-Blutspendedienst-Ost”. Peripheral blood mononuclear cells (PBMC) were isolated by density gradient centrifugation using Ficoll (Biochrom). Cells were cultured in complete medium (RPM11640 [Lonza] with 2 mM UltraGlutamine [Lonza] supplemented with 10 [v/v] fetal calf serum [Linaris], 100 U/ml Penicillin and 100 μg/ml Streptomycin [Lonza]) in flat-bottom plates (PBMC, 6 million cells/ml).

Cells were surface-stained with monoclonal antibodies in PBS containing 10% (v/v) human serum, 2.5% (v/v) FBS and 0.1% (w/v) azide on ice. The following antibodies were used for analysis of pDC activation: anti-lineage cocktail 1, anti-CD123 (7G3), anti-HLA-DR (L243), all from BD Biosciences. All flow cytometric parameters of cells were acquired on a FACSCalibur (BD Biosciences). Mean fluorescence intensity (MFI) of HLA-DR within the pDC fraction was estimated. Data were analyzed with the FlowJo software.

Secreted cytokines were accumulated in cell growth medium for 2 days. ELISA for IFN-alpha (eBioscience), and IP-10 (interferon-inducible protein 10, CXCL10 (R&D Systems)) were performed in duplicates according to the manufacturer's instructions. Optical density was measured at 450 nm; the data were analyzed with the MicroWin software (Berthold Technologies).

Results

FIG. 26: PBMC were stimulated with different TLR-9 agonists comprising at least one nucleotide in L-configuration for 48 hours at a final concentration of 3 μM (A, B, C) or at a concentration range from 0.04 to 3 μM (0). Cell culture medium was used as control. A: Molecules with SEQ ID NO: 5, 7, 8, 10 and 11 were compared to a standard TLR9 agonist; B: Comparison of HLD-DR expression of pDC after stimulation with the indicated molecules; C: Comparison of molecule with SEQ ID NO: 6 with molecule with SEQ ID NO: 5, which was already used for comparison in A; D: Comparison of molecule with SEQ ID NO: 9 with molecule with SEQ ID NO: 5, which was already used for comparison in A. The maximal effects for each molecule within the investigated concentration range are shown.

FIG. 27: PBMC were stimulated with different TLR-9 agonists comprising at least one nucleotide in L-configuration for 48 hours at the indicated concentration range. A: Comparison of molecule with SEQ ID NO: 13 with molecule with SEQ ID NO: 7, which was already used for comparison in FIG. 26A; B: Comparison of molecule with SEQ ID NO: 12 with molecule with SEQ ID NO: 8, which was already used for comparison in FIG. 26A; C: Comparison of molecule with SEQ ID NO: 14 with molecule with SEQ ID NO: 9, which was already used for comparison in FIG. 26D.

It is shown that all tested TLR-9 agonists comprising at least one nucleotide in L-configuration activate TLR9 bearing plasmacytoid cells (pDC) and B cells. IFN-alpha is secreted by pDC and results in a broad activation of the innate and adaptive immune systems. The activation profiles differ between the tested molecules: Individual levels of IFN-alpha obtained after stimulation of PBMC with different molecules vary (FIG. 26A, C, D and FIG. 27), as well as the up-regulation of activation molecules on pDC (FIG. 26B). However, all investigated molecules induced a strong secretion of IP-10 (CXCL10, FIG. 26A, C, D and FIG. 27), a chemokine responsible for the attraction of NK cells and CD8+ T cells to the tumor microenvironment.

The activation of antigen-specific CD8+ T cells, measured by IFN-gamma release was also enhanced (data not shown).

All tested TLR-9 agonists comprising at least one nucleotide in L-configuration this have the potential to modulate the tumor microenvironment.

1. A TLR-9 agonist for use in the treatment of a tumor disease, preferably of colon cancer, in a subject in need thereof, said TLR-9 agonist comprises
i. an oligodeoxyribonucleotide comprising at least one unmethylated CG dinucleotide, wherein C is deoxycytidine and G is deoxyguanosine and
ii. at least one stretch of at least three, in particular of four, consecutive deoxyguanosines,
the deoxyribose moieties of the oligodeoxyribonucleotide are linked by phosphodiester bonds, and the oligodeoxyribonucleotide comprises at least one nucleotide in L-configuration, preferably the at least one nucleotide in L-configuration is comprised within the terminal five nucleotides of at least one end of the oligodeoxyribonucleotide, preferably within the terminal five nucleotides of the 3′ end of the oligodeoxyribonucleotide, wherein the treatment with said TLR-9 agonist stimulates the infiltration of CD3+ T cells into the tumor and/or stimulates the infiltration of macrophages, preferably M1 macrophages, into the tumor and/or leads to an increased ratio of M1 macrophages to M2 macrophages within the tumor.

2. The TLR-9 agonist for use according to claim 1, wherein the infiltration of the tumor by the CD3+ T cells and/or by the macrophages, preferably M1 macrophages, is stimulated compared with the infiltration without the treatment of the TLR-9 agonist and/or the ratio of M1 macrophages to M2 macrophages within the tumor is increased compared to the ratio of M1 macrophages to M2 macrophages without the treatment of the TLR-9 agonist.

3. The TLR-9 agonist for use according to claim 1 or 2, wherein the CD3+ T cells are CD4+ or CD8+ T cells, preferably CD8+ T cells.

4. The TLR-9 agonist for use according to claim 3, wherein the treatment with said TLR-9 agonist leads to an increased frequency of cytotoxic effector T cells (CD8+CD69+ Granzyme B+) within the CD8+ T cell population in the tumor and/or to an increased ratio of CD8+ T cells, preferably of cytotoxic effector T cells (CD8+CD69+ Granzyme B+), to regulatory T cells.

5. The TLR-9 agonist for use according to any of the preceding claims, wherein the tumor is infiltrated in its periphery and/or its center, preferably in its center.

6. The TLR-9 agonist for use according to any of the preceding claims, wherein the tumor is a solid tumor, preferably colon cancer, and the subject to be treated is a human.

7. The TLR-9 agonist for use according to any of the preceding claims, wherein the TLR-9 agonist is administered intratumorally or subcutaneously.

8. The TLR-9 agonist for use according to any of the preceding claims, wherein the at least one CG dinucleotide is part of a sequence N¹N²CGN³N⁴, wherein N¹N² is AA, TT, GG, GT, GA or AT and N³N⁴ is CT, TT, TG or GG and C is deoxycytidine, G is deoxyguanosine, A is deoxyadenosine, and T is deoxythymidine.

9. The TLR-9 agonist for use according to any of the preceding claims, wherein the oligodeoxyribonucleotide comprises at least three CG dinucleotides.

10. The TLR-9 agonist for use according to any of the preceding claims, wherein the oligodeoxyribonucleotide is single-stranded and/or partially or completely double-stranded.

11. The TLR-9 agonist for use according to any of the preceding claims having the sequence of any of SEQ ID NO: 5 to SEQ ID NO: 14, preferably of SEQ ID NO: 5.

12. The TLR-9 agonist for use according to any of the preceding claims, wherein the subject to be treated has previously received and/or subsequently receives another cancer treatment, preferably a chemotherapeutic and/or a checkpoint inhibitor.

13. The TLR-9 agonist for use according to any of the preceding claims, wherein the treatment with said TLR-9 agonist leads to a conversion of a cold tumor into a hot tumor.

14. A TLR-9 agonist for converting a cold tumor into a hot tumor, wherein the TLR-9 agonist comprises
i. an oligodeoxyribonucleotide comprising at least one unmethylated CG dinucleotide, wherein C is deoxycytidine and G is deoxyguanosine and
ii. at least one stretch of at least three, in particular of four, consecutive deoxyguanosines,
the deoxyribose moieties of the oligodeoxyribonucleotide are linked by phosphodiester bonds and the oligodeoxyribonucleotide comprises at least one nucleotide in L-configuration, preferably the at least one nucleotide in L-configuration is comprised within the terminal five nucleotides of at least one end of the oligodeoxyribonucleotide, preferably within the terminal five nucleotides of the 3′ end of the oligodeoxyribonucleotide, and wherein the tumor is preferably colon cancer.

15. A method for increasing the infiltration of CD3+ T cells, preferably CD8+ T cells, into a tumor, preferably colon cancer, and/or for increasing the frequency of cytotoxic effector T cells (CD8+CD69+ Granzyme B+) within the CD8+ T cell population in the tumor and/or for increasing the ratio of CD8+ T cells, preferably of cytotoxic effector T cells (CD8+CD69+ Granzyme B+), to regulatory T cells and/or for increasing the infiltration of macrophages, preferably M1 macrophages, into the tumor and/or for increasing a ratio of M1 macrophages to M2 macrophages within the tumor comprising administering a TLR-9 agonist to a patient in need thereof, wherein the TLR-9 agonist comprises
i. an oligodeoxyribonucleotide comprising at least one unmethylated CG dinucleotide, wherein C is deoxycytidine and G is deoxyguanosine and
ii. at least one stretch of at least three, in particular of four, consecutive deoxyguanosines,
the deoxyribose moieties of the oligodeoxyribonucleotide are linked by phosphodiester bonds and the oligodeoxyribonucleotide comprises at least one nucleotide in L-configuration, preferably the at least one nucleotide in L-configuration is comprised within the terminal five nucleotides of at least one end of the oligodeoxyribonucleotide, preferably within the terminal five nucleotides of the 3′ end of the oligodeoxyribonucleotide.

16. Use of a TLR-9 agonist for increasing the infiltration of CD3+ T cells, preferably CD8+ T cells, into a tumor, preferably colon cancer, and/or for increasing the frequency of cytotoxic effector T cells (CD8+CD69+ Granzyme B+) within the CD8+ T cell population in the tumor and/or for increasing the ratio of CD8+ T cells, preferably of cytotoxic effector T cells (CD8+CD69+ Granzyme B+), to regulatory T cells and/or for increasing the infiltration of macrophages, preferably M1 macrophages, into the tumor and/or for increasing a ratio of M1 macrophages to M2 macrophages within the tumor, wherein the TLR-9 agonist comprises
i. an oligodeoxyribonucleotide comprising at least one unmethylated CG dinucleotide, wherein C is deoxycytidine and G is deoxyguanosine and
ii. at least one stretch of at least three, in particular of four, consecutive deoxyguanosines,
the deoxyribose moieties of the oligodeoxyribonucleotide are linked by phosphodiester bonds and the oligodeoxyribonucleotide comprises at least one nucleotide in L-configuration, preferably the at least one nucleotide in L-configuration is comprised within the terminal five nucleotides of at least one end of the oligodeoxyribonucleotide, preferably within the terminal five nucleotides of the 3′ end of the oligodeoxyribonucleotide.

17. Composition comprising a TLR-9 agonist and a chemotherapeutic and/or a checkpoint inhibitor for use in the treatment of a tumor disease, preferably of colon cancer, in a subject in need thereof, said TLR-9 agonist comprises
i. an oligodeoxyribonucleotide comprising at least one unmethylated CG dinucleotide, wherein C is deoxycytidine and G is deoxyguanosine and
ii. at least one stretch of at least three, in particular of four, consecutive deoxyguanosines,
the deoxyribose moieties of the oligodeoxyribonucleotide are linked by phosphodiester bonds and the oligodeoxyribonucleotide comprises at least one nucleotide in L-configuration, preferably the at least one nucleotide in L-configuration is comprised within the terminal five nucleotides of at least one end of the oligodeoxyribonucleotide, preferably within the terminal five nucleotides of the 3′ end of the oligodeoxyribonucleotide, wherein the treatment with said TLR-9 agonist stimulates the infiltration of CD3+ T cells into the tumor and/or stimulates the infiltration of macrophages, preferably M1 macrophages, into the tumor and/or leads to an increased ratio of M1 macrophages to M2 macrophages within the tumor.

18. A pharmaceutical composition comprising 1 mg/ml to 30 mg/ml, preferably 10 mg/ml to 20 mg/ml, of a TLR-9 agonist comprising at least one nucleotide in L-configuration in glucose in a salt solution.
